(19) United States
(12) Patent Application Publication
(10) Pub. No.: US 2010/0168621 A1
(43) Pub. Date: Jul. 1, 2010

(54) METHODS AND SYSTEMS FOR PROSTATE HEALTH MONITORING

(76) Inventor: Thomas B. Neville, Incline Village, NV (US)

Correspondence Address: WILSON, SONSINI, GOODRICH & ROSATI, 650 PAGE MILL ROAD, PALO ALTO, CA, 94304-1050, US

(21) Appl. No.: 12/645,002

(22) Filed: Dec. 22, 2009

Related U.S. Application Data
(60) Provisional application No. 61/140,581, filed on Dec. 23, 2008.

(51) Int. Cl. A61B 5/103; A61M 31/00

(52) U.S. Cl. 600/587; 604/500

(57) ABSTRACT

Disclosed herein are computer-implemented personalized probabilities determination systems and methods for use in integrated health systems and methods related to the medical conditions, such as conditions of the prostate, including benign and malignant disease. A system and method is disclosed herein for estimating trends in biomarkers and calculating the probability of different prostate medical conditions. Also disclosed herein are methods and systems relating to estimating prostate volume and probabilities of benign prostatic hyperplasia (BPH). Methods and systems relating to probabilities and feedback during treatment of prostatic conditions or diseases are also provided herein.

### CROSS-REFERENCE

This application claims the benefit of U.S. Provisional Application No. 61/140,581 filed Dec. 23, 2008, which application is incorporated herein by reference.

### BACKGROUND OF THE INVENTION

An emphasis on early detection and treatment and therefore avoiding unnecessary treatment and invasive procedures has been increased in recent years. Significant investments are being made to accelerate discovery and use of biomarkers that effectively detect progressing and existing diseases. However, assaying or testing for a single biomarker is often not effective for detection of progressing disease.

The use of screening blood tests, where multiple markers are tested, is becoming more prevalent and cost-effective. Screening for many conditions using blood from a single draw can reduce medical costs. The incremental cost of additional tests decreases if subsequent blood draws are not needed. A further means of reducing costs is to store blood for later testing if needed. New technology is also reducing the cost of specific tests.

Prostatic disease is likely to occur in every man at some point in his life. Increased research on prostate health, including disease such as benign prostate hyperplasia (BPH), prostatitis, and prostate cancer has been occurring in medicine in the last few years. However, due to the interrelations of the prostatic diseases, it is difficult to treat and diagnose prostate disease in non-invasive fashions. In addition, individual biomarker assays, such as prostate specific antigen (PSA), can be inaccurate when due to the interrelations when trying to detect an individual disease.

There is a need in the art to extract additional information from a prostate diagnostic test, whether it is a biomarker test or a series of biomarker tests, or a medical image. Novel methods and systems of extracting additional quantitative information for use by patients or physicians are increasingly desirable to reduce the cost of medical diagnostics and treatments and to improve the accuracy of diagnosis and efficacy of prostate treatments.

### SUMMARY OF THE INVENTION

In an aspect, a method of detecting prostate enlargement comprises: monitoring a prostate biomarker value at one or more times; measuring a volume of the prostate of the subject when the prostate biomarker value is or exceeds a target value; calibrating the prostate biomarker value with the volume measurement; and detecting the presence or absence of prostate enlargement in the subject by measuring the prostate biomarker value. In some instances, the calibrating step comprises determining the prostate biomarker value density. In an embodiment, the prostate biomarker is PSA or fPSA. In some instances, the method can further comprise monitoring the prostate biomarker value two or more times, thereby forming a prostate biomarker trend. A monitoring step can comprise eliminating anomalous prostate biomarker values from the trend that are values outside of a tolerance. In some instances, the measuring step comprises an ultrasound measurement. In yet other instances, the method can further comprise calculating the probability of prostate enlargement from the prostate biomarker value.

In another aspect, a method of performing a course of medical action for prostate enlargement in a subject is disclosed, the method comprising: monitoring a prostate biomarker value from a subject at one or more times; measuring a volume of the prostate of the subject when the prostate biomarker value is or exceeds a target value; calibrating the prostate biomarker value with the volume measurement; estimating the volume of the prostate by measuring the prostate biomarker value at a time after the measuring step; performing a course of medical action when the estimated volume of the prostate is greater than a threshold value. The method can further comprise repeating the estimating and performing steps after determining the outcome of the course of medical action. In an embodiment, the calibrating and estimating are performed by a computer system. A computer system can comprise a device for network communication, a storage unit, and a processor. The computer system can comprise a Monte Carlo engine. In some instances, the method further comprises calculating the probability of prostate enlargement from the prostate biomarker value. In some instances, the calibrating step comprises determining the prostate biomarker value density. In an embodiment, the prostate biomarker is PSA or fPSA. In some instances, a method comprises monitoring the prostate biomarker value two or more times, thereby forming a prostate biomarker trend. The monitoring step can comprise eliminating anomalous prostate biomarker values from the trend that are values outside of a tolerance. In some instances, a measuring step comprises an ultrasound measurement. A course of medical action can be delivering medical treatment to said subject, such as a pharmaceutical, TURP, TUNA, or TUMT. A course of medical action can comprise administration of medical tests, medical imaging of said subject, and/or consultation with a medical professional. In some instances, a method further comprises performing a digital rectal exam and calibrating the prostate biomarker value with the digital rectal exam results and the volume measurement.

In an aspect, a method is disclosed comprising: establishing a Bayesian network comprising a plurality of prostatitis conditions of the prostate, wherein the priors of the Bayesian network are provided by historical data; calculating a probability of a target prostatitis condition of a prostate using a trend of a prostate biomarker value over time; providing the probability of the target medical condition of the prostate to the Bayesian network as a prior for the target medical condition; performing a course of medical action in respect to the target medical condition and receiving the result of the course of medical action; adjusting the priors of the Bayesian network according to the result; and calculating the probability of the target prostatitis condition. The target prostatitis condition can be inflammation or infection. In some instances, the course of medical action is delivering an anti-inflammatory, delivering an anti-biotic, performing a white blood cell count, and/or culturing prostate secretions. In an embodiment, a method further comprises detecting a change in the prostate biomarker value after step performing a course of medical action. The change can demonstrate an effectiveness of a course of medical action. The method can further comprise calculating the probability of prostate cancer using the probability of the target prostatitis condition.

In another aspect, a method of providing a medical treatment of a prostate condition is disclosed that comprises: providing a course of medical action to a subject with a prostate condition; detecting a change in a prostate biomarker value of the subject before providing the medical treatment and during the medical treatment; and adjusting the course of medical action based upon the change in the prostate biomarker value. The prostate biomarker can be PSA or fPSA. A method can further comprise detecting a change in the prostate biomarker value at more than one time during treatment. In some instances, the prostate biomarker over time forms a prostate biomarker trend and wherein the change in the prostate biomarker value is detected by a change in the prostate biomarker trend. The prostate condition can be BPH and the course of medical action can comprise delivering a pharmaceutical to the subject, and the adjusting of the course of medical action can comprise adjusting at least one of the following: treatment time or treatment dose. The prostate condition can be prostatitis infection and the course of medical action can be delivering an antibiotic to the subject, and the adjusting of the course of medical action can comprise adjusting at least one of the following: treatment time or treatment dose. The prostate condition can be prostatitis inflammation and the course of medical action can be delivering an anti-inflammatory to the subject, and the adjusting of the course of medical action can be adjusting at least one of the following: treatment time or treatment dose. In some instances, the course of medical action is delivering a pharmaceutical to the subject, and the adjusting of the course of medical action comprises changing the pharmaceutical being delivered during treatment.

### INCORPORATION BY REFERENCE

All publications mentioned in this specification are herein incorporated by reference to the same extent as if each individual publication was specifically and individually indicated to be incorporated by reference.

### BRIEF DESCRIPTION OF THE DRAWINGS

Many features of the invention are set forth with particularity in the appended claims. A better understanding of the features and advantages of the invention will be obtained by reference to the following detailed description that sets forth illustrative embodiments, in which many principles of the invention are utilized, and the accompanying drawings of which:

FIG. 1 demonstrates an example wherein very minor symptoms begin to appear as the prostate grows larger than roughly 50 cc.

FIG. 2 shows an example of the enlargement process and age for a man.

FIG. 3 shows an example of a man with BPH whose symptoms reach a high level at age 70 when he is treated with combination medication.

FIG. 4 shows results of late treatment for the man in FIG. 3.

FIG. 5 illustrates a BPH volume threshold is the prostate volume at which early treatment should be considered.

FIG. 6 shows the relationship for an example man wherein PSA tends to increase as prostate volume increases.

FIG. 7 shows a range of PSA densities for men.

FIG. 8 shows the example 60 cc volume threshold (dashed line).

FIG. 9 shows estimates of the PSA trend based on a high PSA density (0.05), median density (0.04) and low density (0.03).

FIG. 10 illustrates volume trends that can be estimated that correspond to the true PSA trend using a range of PSA densities.

FIG. 11 shows estimated volume trends for low PSA density (0.03), median density (0.04) and high density (0.05).

FIG. 12 shows the low density volume trend reaches the 60 cc threshold at age 56.

FIG. 13 shows a possible true PSA trend projection through age 80.

FIG. 14 shows that at age 64 the second volume measurement confirms that the man's prostate volume has reached the 60 cc threshold.

FIG. 15, illustrates the start of mild doses of two combined medicines that last for the rest of the man's life.

FIG. 16 shows an example wherein moderate dose treatment can shrink prostate volume enough to avoid reaching the volume threshold for a reasonable number of years after treatment.

FIG. 17 demonstrates an exemplary simplified version of the Bayesian network as described herein for the prostatitis detection process.

FIG. 18 shows a Bayesian network.

FIG. 19 shows an example wherein a man with a healthy prostate that produces 1.0 of measured PSA at age fifty.

FIG. 20 shows the start of a course of antibiotic treatment using a standard dose of a typical antibiotic.

FIG. 21 shows PSA trends for effective treatment.

FIG. 22 shows the start of a course of antibiotic treatment using a high dose of a typical antibiotic.

FIG. 23 demonstrates an example wherein a man with a healthy prostate produces 1.0 of measured PSA at age fifty and soon after, mild inflammation starts to affect his prostate with growing severity.

FIG. 24 shows the start of a course of treatment using a standard dose of a typical anti-inflammatory medication.

FIG. 25 shows the probability of two temporary benign conditions for anomalous test results over time for a man.

FIG. 26 illustrates an example wherein, prior probabilities may be a function of age, race, genetics, demographics, past experience with the conditions and other considerations as shown in.

FIG. 27 and FIG. 28 show dynamic screening probability generator for temporary prostate conditions consolidates output from three separate probability generators: inflammation prostatitis, infection prostatitis and other temporary conditions.

FIG. 29 shows the probability distributions of a prostate condition that can be affected by past medical experience with the conditions, and the results of imaging, tests, treatment and other medical procedures.

FIG. 30 illustrates an example wherein a positive bacterial culture and/or beneficial impact of antibiotic treatment may increase the probability of infection prostatitis to a high level and reduce the probability of inflammation and the probability of other conditions.

FIG. 31 illustrates an exemplary embodiment, wherein four similar Bayes processes are used to calculate the probability of the prostate conditions: volume growth due to BPH, inflammation prostatitis, infection prostatitis and progressing cancer.

FIG. 32 shows the probability distributions of prostate conditions affected by past experience and the results of imaging, tests, treatment and other medical procedures.

FIG. 33 demonstrates an example computer system of the invention wherein a user can use a web browser to enter data from a prostate biomarker assay into a graphical user interface of a webpage.

FIG. 34 illustrates a method of delivering a probability that a subject has a medical condition to a user and using the probability to take a course of medical action.

FIG. 35 demonstrates an exemplary method and system of the invention for estimating volume of a prostate.

FIG. 36 illustrates exemplary volume measurements using solid lines connecting volume measurements.

FIG. 37 demonstrates equilibration of PSA after treatment within a few weeks or months.

### DETAILED DESCRIPTION OF THE INVENTION

Methods, business methods, and systems are provided herein for integrated healthcare systems for prostate health. As the amount of information increases rapidly, such as information from multiple biomarkers, analysis and management becomes more important in order to to extract meaningful conclusions from the information. Methods and systems, as described herein, provide calculations of prostate biomarker values into analytical data for a user. The methods and systems are described herein in the context of analyzing data from men regarding prostate medical conditions.

Disclosed herein are computer-implemented personalized probabilities determination systems and methods for use in integrated health systems and methods related to the medical conditions, such as conditions of the prostate, including benign and malignant disease. For example, a system and method is disclosed herein for estimating trends in biomarkers and calculating the probability of different prostate medical conditions. Also disclosed herein are methods and systems relating to estimating prostate volume and probabilities of benign prostatic hyperplasia (BPH). Methods and systems relating to probabilities and feedback during treatment of prostatic conditions or diseases are also provided herein.

### Benign Prostatic Hyperplasia (BPH)

In an aspect, a detection system is disclosed for identifying an enlarging prostate caused by BPH. In some instances, a detection system herein can allow for early treatment. In another aspect, a treatment monitoring and optimization system is disclosed to monitor the effect of BPH treatments to alter dosage and choice of medications over time and to determine when treatment should be stopped or resumed.

Benign prostatic hyperplasia (BPH) refers to the increase in size of the prostate in middle-aged and elderly men. It is characterized by hyperplasia of prostatic stromal and epithelial cells, resulting in the formation of large, fairly discrete nodules in the periurethral region of the prostate. When sufficiently large, the nodules compress the urethral canal to cause partial, or sometimes complete, obstruction of the urethra which interferes the normal flow of urine. It leads to symptoms of urinary hesitancy, frequent urination, increased risk of urinary tract infections and urinary retention. Prostate specific antigen (PSA) levels may be elevated in these patients because of increased organ volume and inflammation due to urinary tract infections.

BPH symptoms can be classified as storage or voiding. Storage symptoms include urinary frequency, urgency (compelling need to void that can not be deferred), urgency incontinence and voiding at night (nocturia).

BPH can be a progressive disease, especially if left untreated. Incomplete voiding results in stasis of bacteria in the bladder residue and an increased risk of urinary tract infections. Urinary bladder stones, are formed from the crystallisation of salts in the residual urine. Urinary retention, termed acute or chronic, is another form of progression. Acute urinary retention is the inability to void, while in chronic urinary retention the residual urinary volume gradually increases, and the bladder distends. Some patients who suffer from chronic urinary retention may eventually progress to renal failure, a condition termed obstructive uropathy.

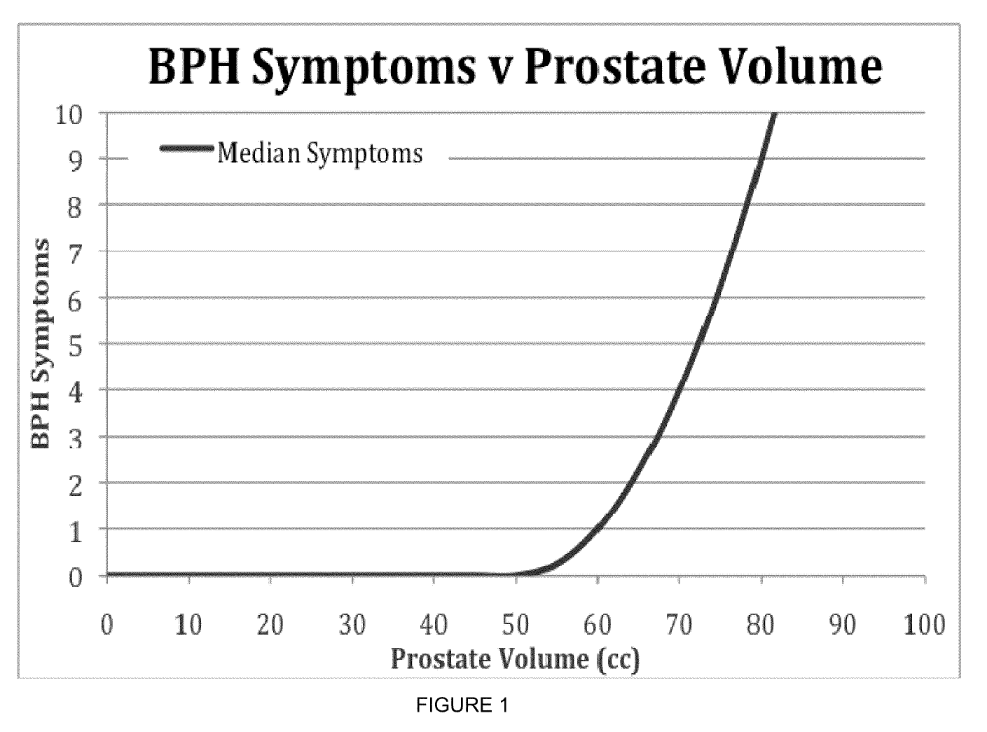

In many instances, healthy prostates for younger men have a volume of about 25 cc. BPH causes prostates to enlarge for most men as they age. BPH symptoms are typically not present until the prostate becomes large enough to impinge on a man's urinary system. In the example of FIG. 1, very minor symptoms begin to appear as the prostate grows larger than roughly 50 cc. Additional volume growth leads to symptoms that grow at an increasingly rapid rate. The symptom scale on the vertical axis is suggestive and does not reflect a specific symptom severity measurement method.

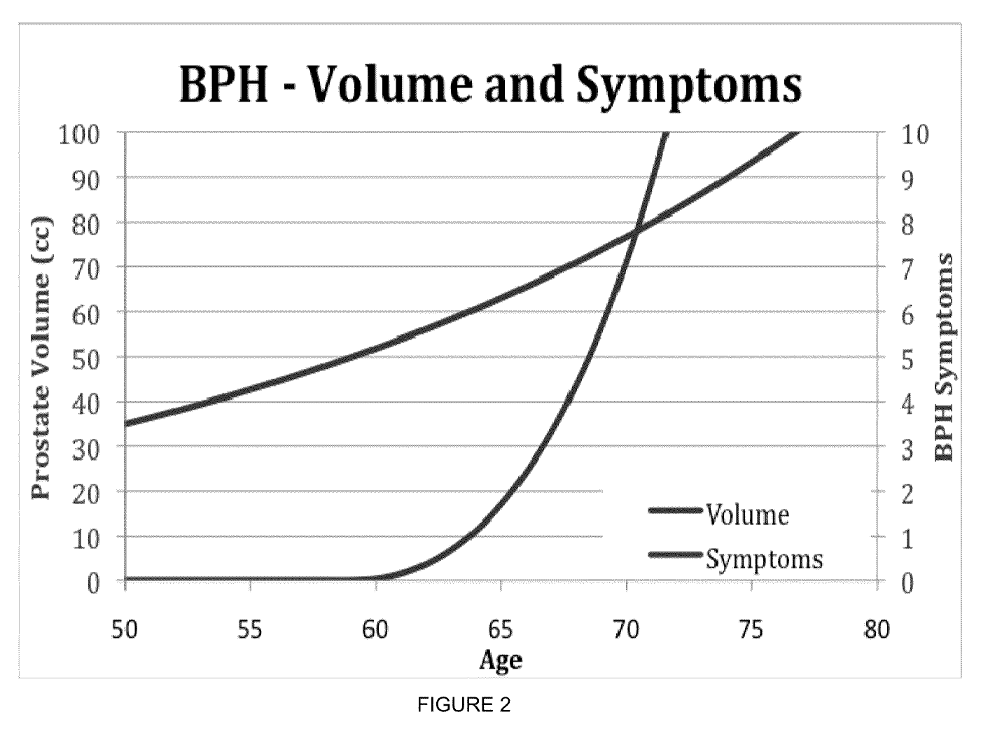

For most men, prostate volume grows as they age. For some men, prostates grow large enough for symptoms to appear and sometimes the symptoms become severe. FIG. 2 shows an example of the enlargement process and age for a man. The gradual curve shows volume growth relative to the left scale. The more severe curve shows symptom severity relative to the right scale.

Example treatments for BPH include lifestyle changes such as decreasing fluid intake before bedtime, moderate the consumption of alcohol and caffeine-containing products, and timed voiding schedules. Other treatment options include pharmaceuticals such as Alpha blockers to provide symptomatic relief of BPH symptoms. Available drugs for prostate symptoms include doxazosin, terazosin, alfuzosin and tamsulosin. The 5α-reductase inhibitors (finasteride dutasteride) are another treatment option. In embodiments herein, a method can comprise delivering a drug for relieving BPH symptoms as described herein.

While medication is often prescribed as the first treatment option in many instances, there are many patients who do not achieve success. In some instances, a thermal therapy may be used to treat BPH, such as transurethral microwave thermotherapy (TUMT) and transurethral needle ablation (TUNA). The goal of the therapies is to cause enough necrosis so that when the dead tissue is reabsorbed by the body the prostate shrinks, relieving the obstruction of the urethra. In some instances, methods herein comprise delivering thermal therapy to a patient exhibiting BPH, or having biomarker values indicating BPH.

Another course of medical action for BPH is transurethral resection of prostate (TURP). This involves surgically removing part of the prostate through the urethra. Other treatments and courses of medical action can be or are being developed for treatment of medical conditions of the prostate. A course of medical action as required for some embodiments herein can include surgery, such as TURP. Post surgery care often involves placement of a Foley Catheter or a temporary Prostatic stent to allow healing and urine to drain from the bladder.

Men with very large prostates and severe symptoms often need surgery or outpatient methods of killing prostate cells and shrinking the prostate. In some instances, a course of medical action for men with milder symptoms from slightly enlarged prostates can be treated effectively with a combination of two medications, 5α-reductase inhibitors (such as finasteride dutasteride) and alpha blockers (such as doxazosin, terazosin, alfuzosin and tamsulosin), which reduce prostate size and symptoms 66% of the time. (Kaplan S A, McConnell J D, Roehrborn C G, et al (2006). “Combination therapy with doxazosin and finasteride for benign prostatic hyperplasia in patients with lower urinary tract symptoms and a baseline total prostate volume of 25 ml or greater”. J. Urol. 175 (1): 217-20; discussion 220-1.)

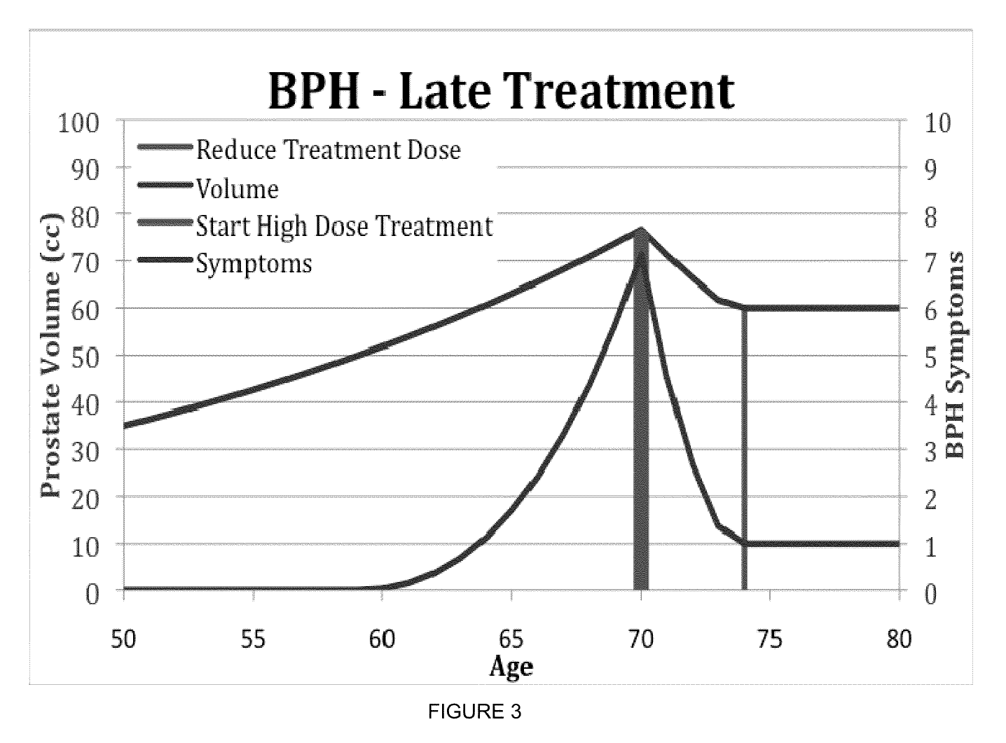

FIG. 3 shows an example of a man with BPH whose symptoms reach a high level at age 70 when he is treated with combination medication. The doses are high enough to be effective but cause side effects (the thick vertical bar). Over time the man's prostate decreases in size and symptoms decrease to an acceptable level. At that point treatment doses can be reduced, as shown by the thin red bar, or eliminated until prostate volume increases enough in the future to warrant another round of treatment.

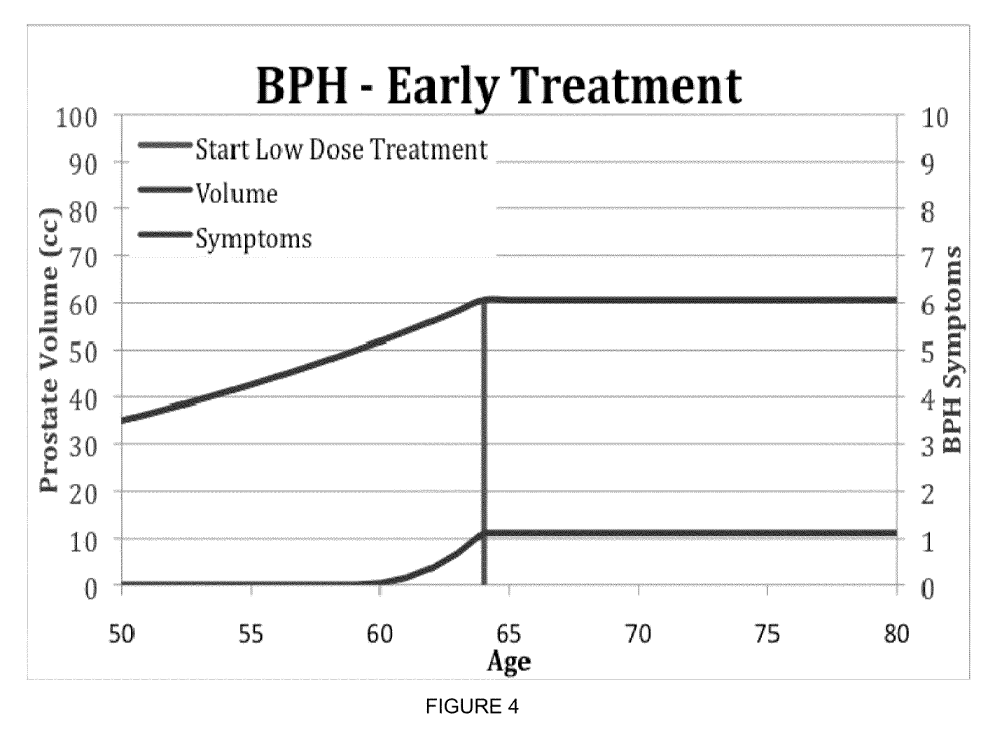

The methods and systems described herein can pertain to early treatment of BPH before symptoms become severe, or even noticed. FIG. 4 shows results for the man used in the previous example of late treatment. Prostate volume reaches a threshold of concern at age 64 often before symptoms are noticed and, in cases where symptoms are noticed, before they become severe. Low dose treatment will produce milder side effects than high dose treatment required for late detection of BPH.

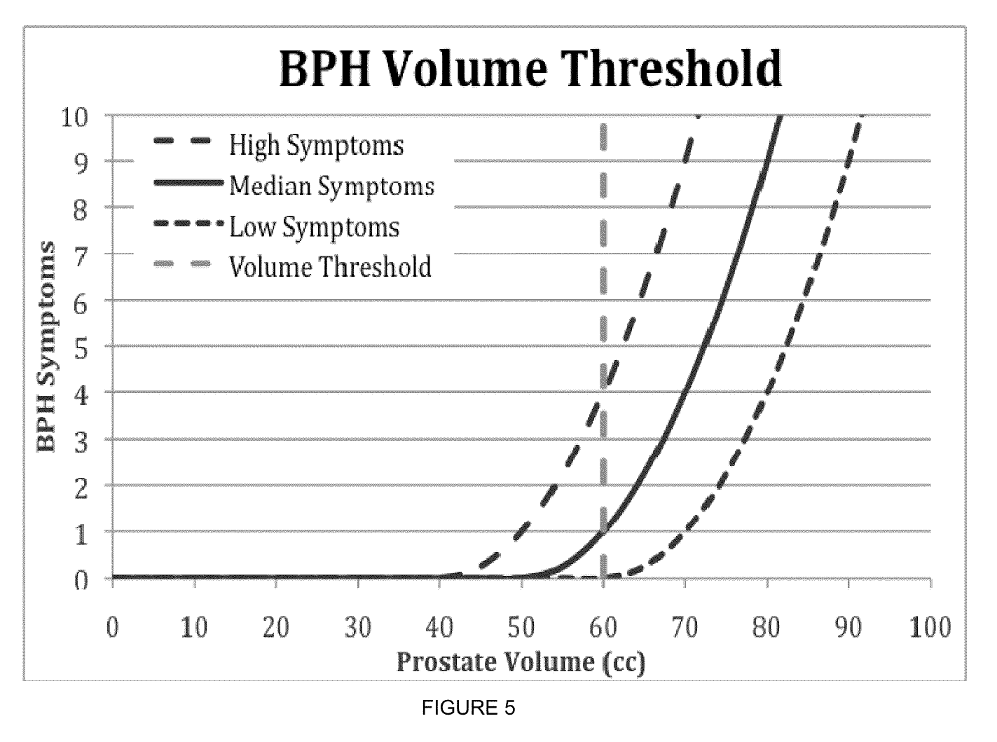

As described herein, BPH volume threshold is the prostate volume at which early treatment should be considered. For example in FIG. 5, it is shown by the vertical dashed line. The threshold can be set to a variety of values according to the user or medical personnel. Some men will have symptoms at smaller prostate volumes (dashed curve) and some will have symptoms at larger prostate volumes (dotted curve).

Some BPH symptoms can be caused by prostatitis, progressing cancer and other conditions. Therefore in some instances, medical treatments are not always necessary when BPH symptoms are present.

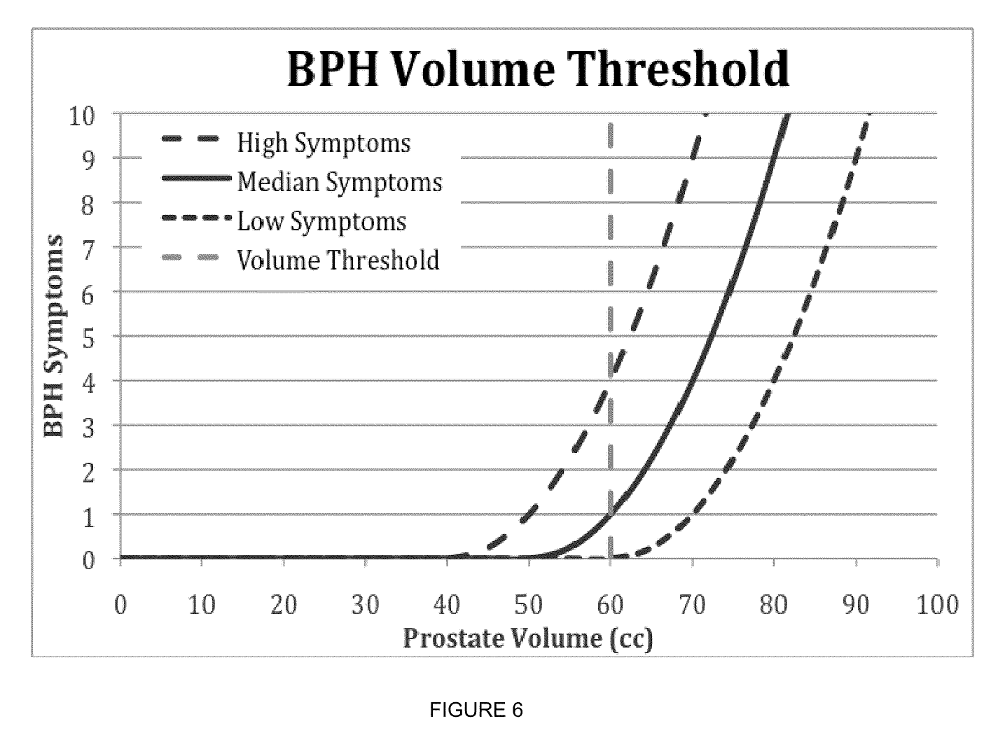

In many instances, PSA tends to increase as prostate volume increases. FIG. 6 shows the relationship for an example man. The slope of the line is determined by the PSA density of the prostate, in this case the median density. Higher density means a steeper slope, and lower density means a flatter slope.

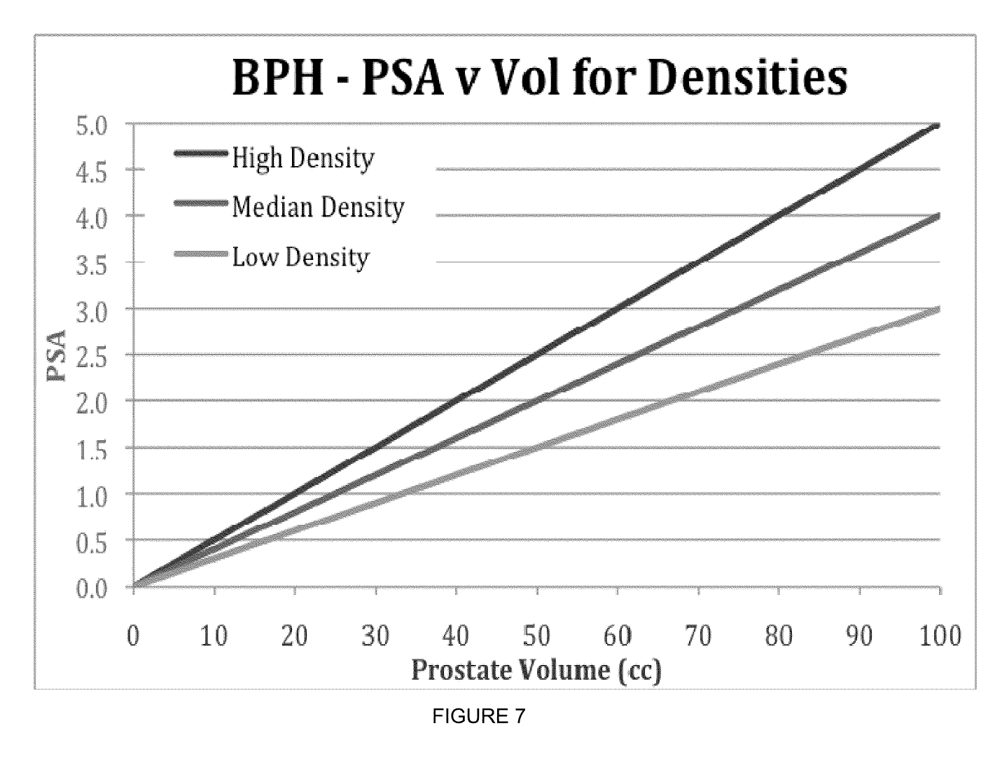

There can be a range of PSA densities for men. The dark line on FIG. 7 shows high PSA density. The light line shows low PSA density.

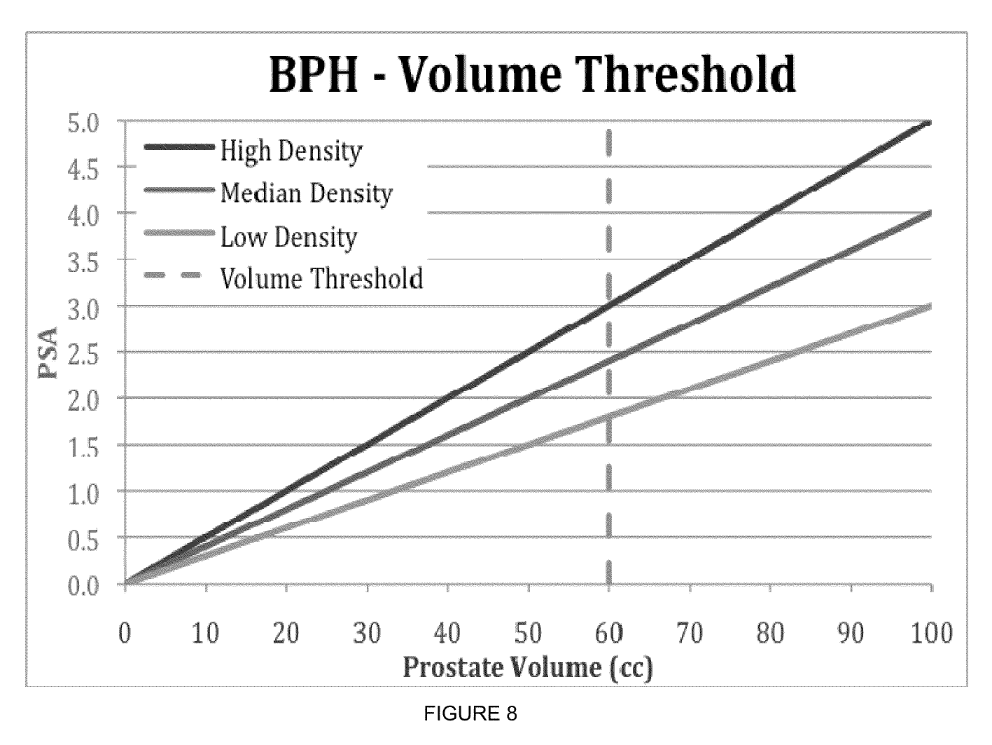

FIG. 8 shows the example 60 cc volume threshold (dashed line). It intersects the three lines at different levels of PSA. The equivalent PSA threshold is higher for a high density prostate and lower for a low density prostate.

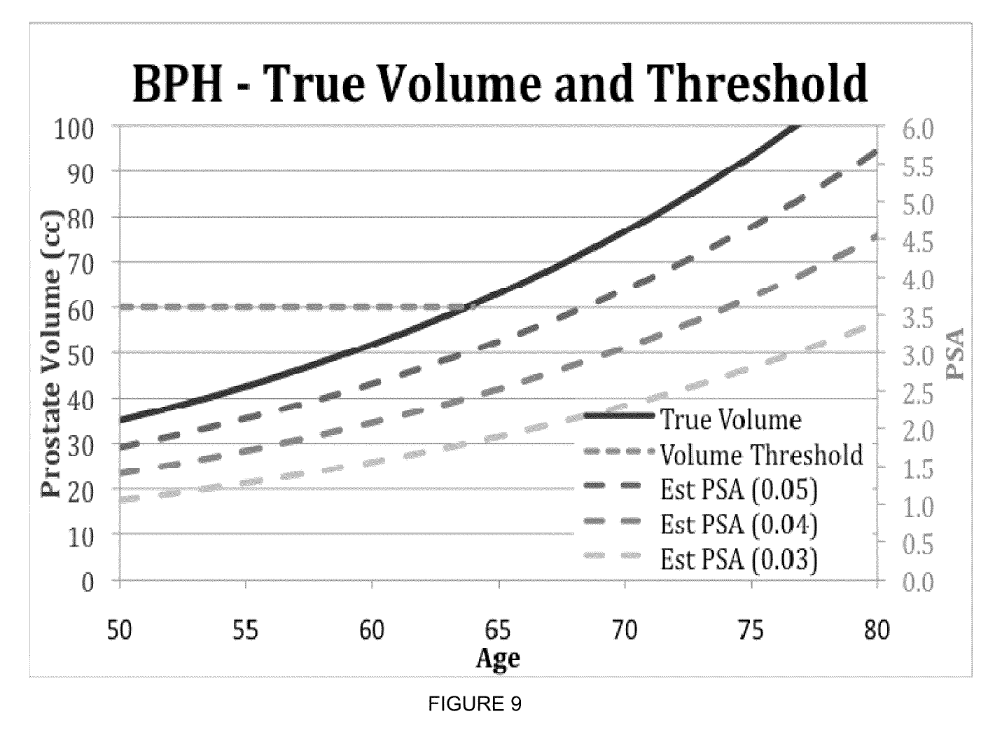

In some embodiments, PSA trends can be estimated corresponding to the true volume trend using a range of PSA densities. FIG. 9 shows estimates of the PSA trend based on a high PSA density (0.05), median density (0.04) and low density (0.03). PSA values are read off the scale on the right side.

FIG. 9 shows that an exemplary volume threshold of 60 cc is reached at age 64. Each PSA density causes its PSA trend to reach a different level at age 64 when true volume reaches the threshold.

In many cases, a subject monitors a PSA trend and does not have measurements of prostate volume. In an embodiment, a PSA trend, (for example change in PSA over time), can be estimated by measuring the PSA biomarker values at different times and eliminating anomalous values. In addition, probabilities of other prostate conditions, such as cancer and prostatitis can be identified in order to better relate an increasing PSA trend with volume growth of a prostate.

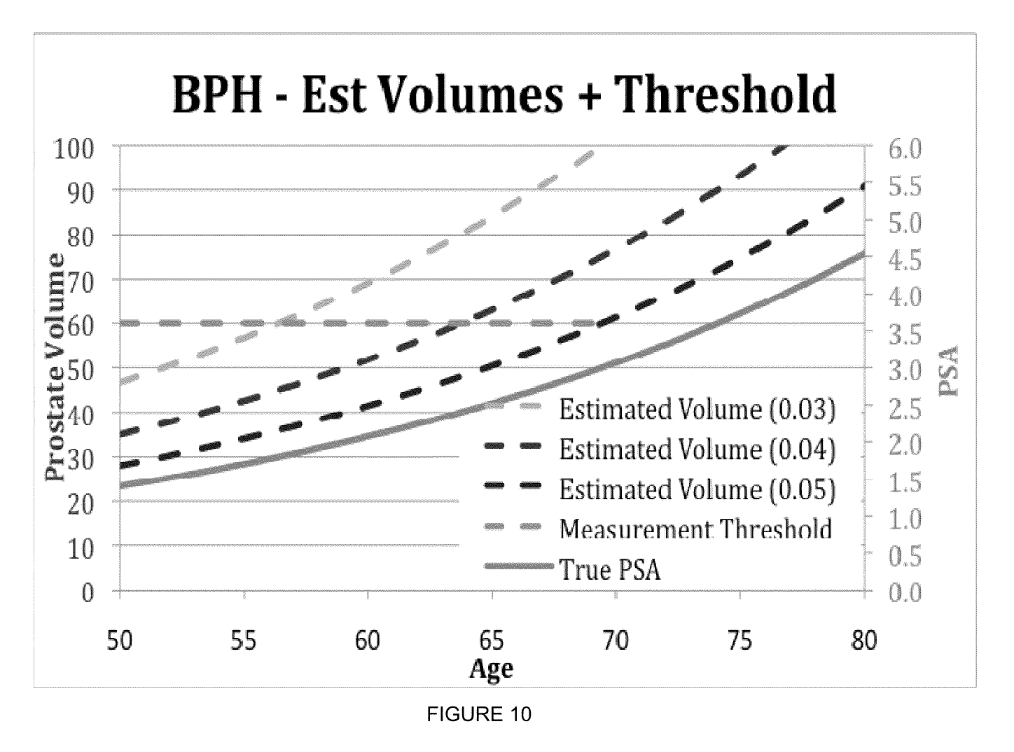

In the example of FIG. 10, volume trends can be estimated that correspond to the true PSA trend using a range of PSA densities. FIG. 10 shows estimates of the volume trend based on a low PSA density (0.03), median density (0.04) and high density (0.05). PSA values are read off the scale on the right side. FIG. 10 shows that an exemplary volume threshold of 60 cc is reached at age 56 for low PSA density, at age 64 for median PSA density and at age 69 for high PSA density.

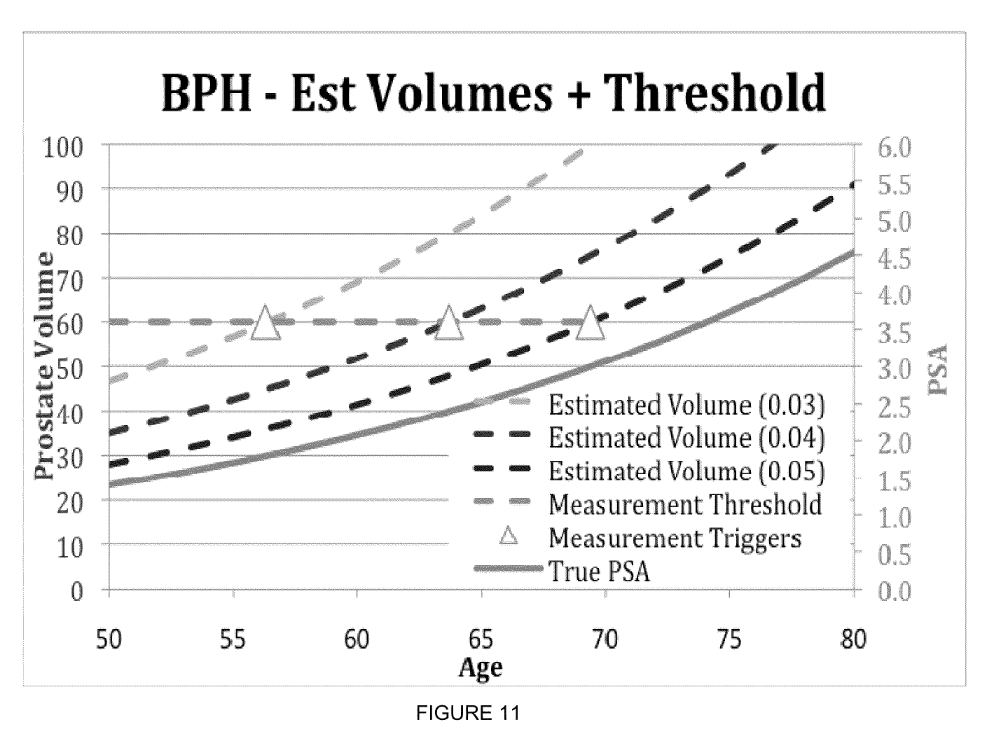

Many men do not have a prostate volume measurement to help estimate their PSA density. For them, there is a range of estimated volume trends based on a range of possible PSA densities. In FIG. 11, the estimated volume trends are shown as dashed curves: light for low PSA density (0.03), bright for median density (0.04) and dark for high density (0.05). Each estimated volume trend reaches the 60 cc volume threshold at different ages: 56 for low density, 64 for median density and 69 for high density—as shown by the three triangles.

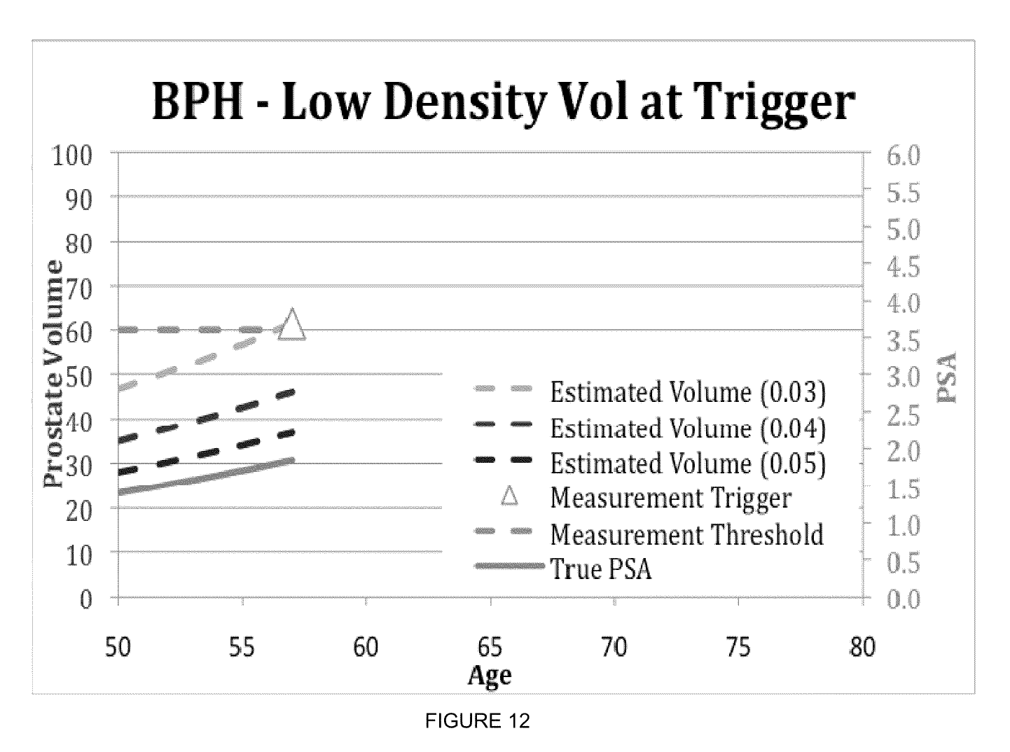

In an example, a BPH screening method as described herein is to trigger a volume measurement when the low density volume trend reaches the volume threshold. The triangle in FIG. 12 shows the low density volume trend reaches the 60 cc threshold at age 56. The low density volume trend can be an early indicator that prostate volume might be large enough to reach the threshold. When the threshold is reached, a volume measurement is conducted using a method herein to determine the density of the prostate.

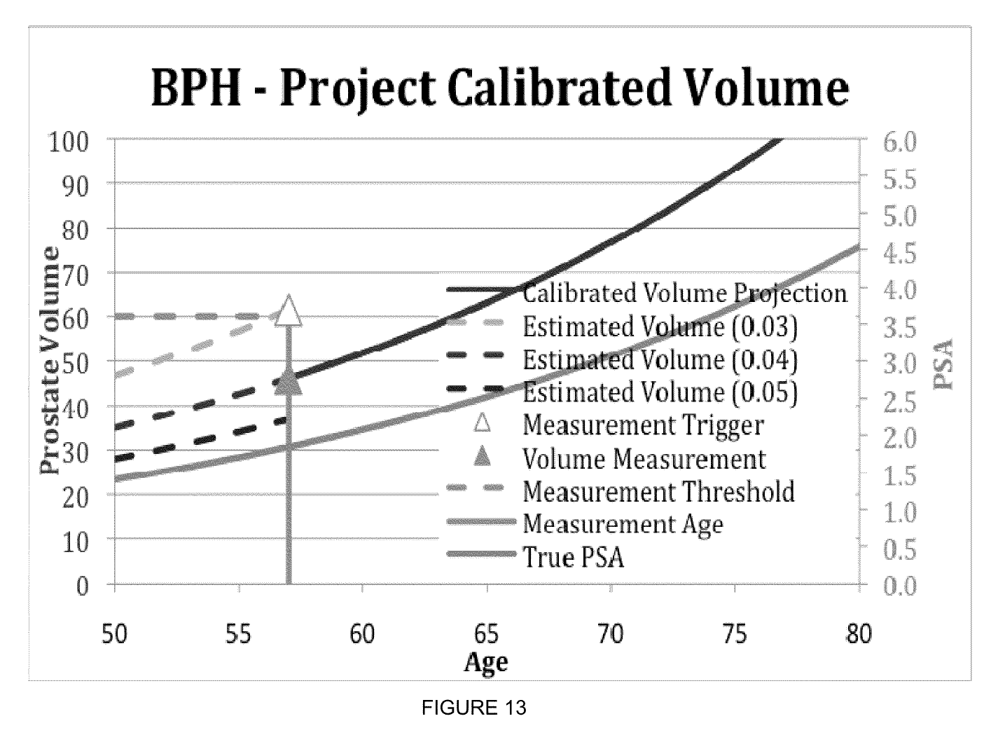

In an example of the figures, a volume measurement is triggered at age 56 by the low density volume trend, as shown by the solid vertical line in FIG. 13. In this example the volume measurement is about 45 cc, as shown by the solid triangle in FIG. 13. This measurement and the PSA trend at age 56 allow us to calculate the PSA density as 0.04, the median value.

In an embodiment, the PSA density of 0.04 shows with more confidence to estimate a calibrated volume trend based on a true PSA trend. The bottom curve in FIG. 13 shows a possible true PSA trend projection through age 80. The top curve shows the corresponding estimated volume trend calibrated using the 0.04 PSA density at age 56.

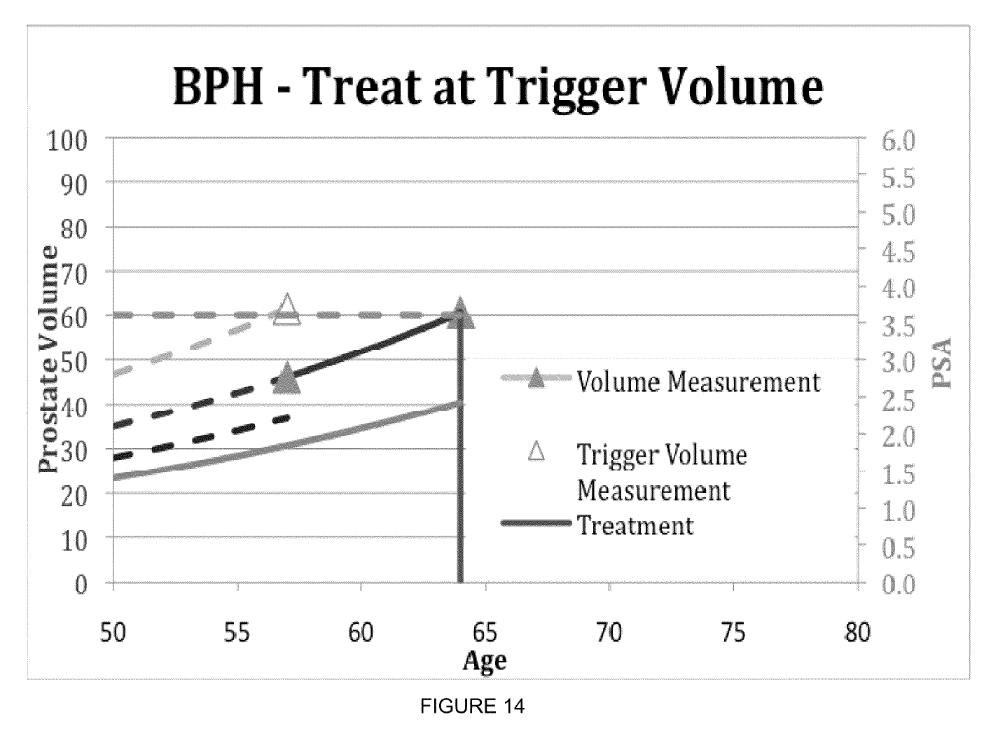

In FIG. 14, the solid triangle at age 64 shows that the second volume measurement confirms that the man's prostate volume has reached the 60 cc threshold. The vertical line at age 64 in FIG. 14 indicates the start of treatment for BPH.

When choosing a course of medical action, two exemplary methods are described in this section comprising: long-term treatment to prevent volume growth and moderate temporary treatment to reduce volume.

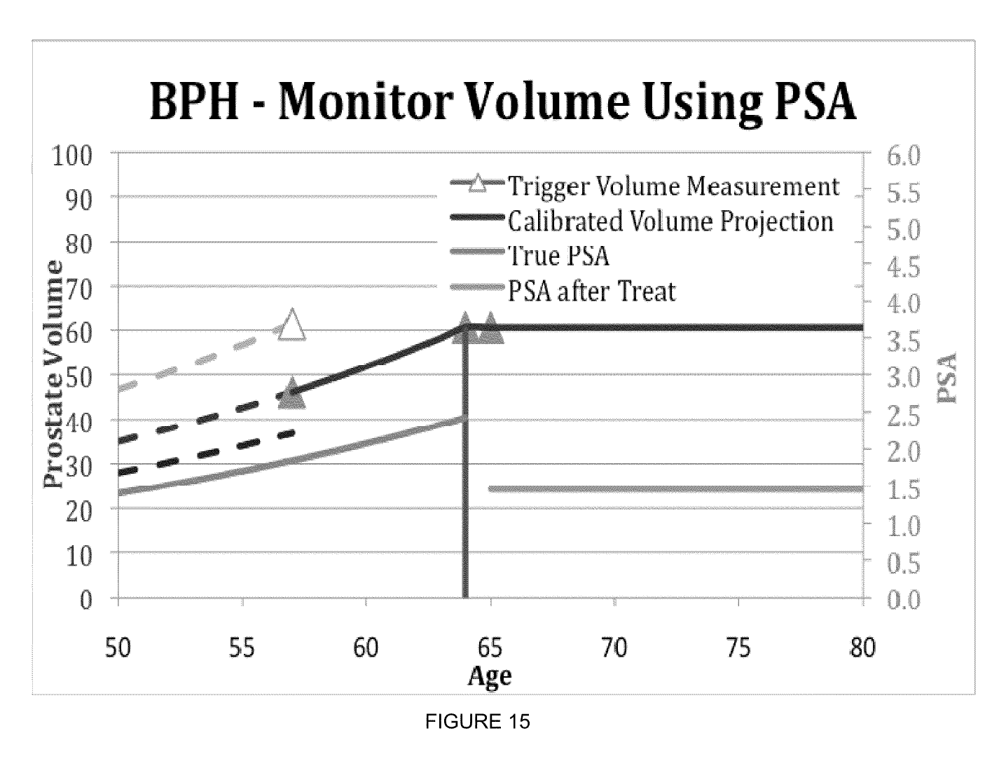

In FIG. 15, the vertical line indicates the start of mild doses of two combined medicines that last for the rest of the man's life. The dotted line after age 64 shows the expected constant prostate volume if the dosage is appropriate for this exemplary strategy. As an example, full dose treatment using a 5α-reductase inhibitor (finasteride dutasteride) can reduce PSA by as much as 50% or more. The amount of reduction from a reduced mild dose to limit further volume growth will be less but is still likely to be significant. The dark curve in FIG. 15 shows the PSA trend prior to mild treatment for BPH. Treatment starts at age 64, as shown by the vertical line, and continues through age 80 on the graph below. The figure shows the reduction in PSA caused by the continuing treatment. After treatment has had time to affect PSA fully, a second calibrating volume measurement will be suggested—at age 65 in the example, shown by the last solid triangle. This new measurement may confirm that prostate volume remains constant, as planned. In some instances, dose can be increased if volume increases and reduced if volume decreases. The new measurement also allows a new, post medication calculation of PSA density in conjunction with the new stable PSA trend. The new density can be used in conjunction with the future PSA trend to monitor estimated PSA volume, shown by the solid line after age the last volume measurement at age 65. If estimated volume based on PSA increases significantly over time a new volume measurement can establish whether volume has actually increased and treatment dose should be increased or volume has not increased but PSA density has decreased and should be recalibrated.

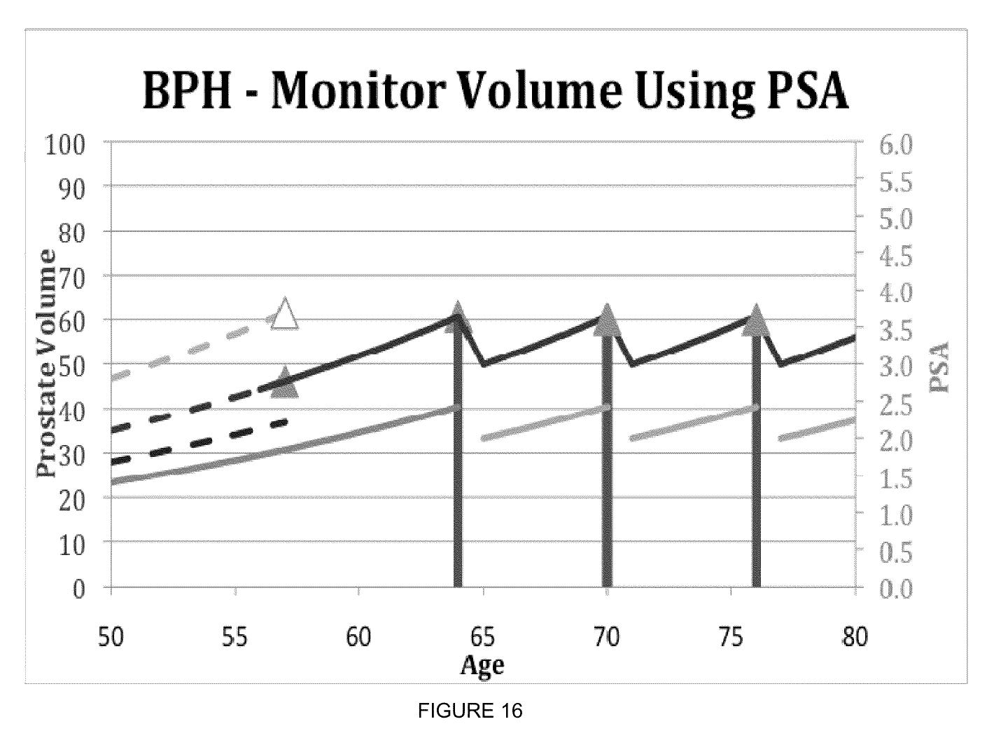

In another example, as shown in FIG. 16, moderate dose treatment can shrink prostate volume enough to avoid reaching the volume threshold for a reasonable number of years after treatment. Compared to full dose treatment after significant symptoms caused by a large prostate, this approach reduces the severity of side effects and can reduce the treatment period over which those side effects are endured. FIG. 16 shows the first treatment starting at age 64, as before, but lasting only a year, for example, because the moderate dose is larger than the mild dose intended to prevent further growth. The larger moderate dose shrinks the prostate from 60 cc at age 64 to 50 cc by age 65 when the treatment is stopped. After treatment stops PSA density returns to pre treatment levels. However, PSA drops after treatment is stopped at age 65 because the prostate is smaller than it was before treatment. Growth in prostate volume can be estimated using past PSA density and the PSA trend from age 65 to age 70. Estimated volume reaches the 60 cc threshold at age 70. A volume measurement is triggered at that age, which confirms the 60 cc volume. A second round of moderate treatment is administered from age 70 to 71 when it is stopped. Prostate volume again drops to 50 cc and PSA drops accordingly. The process repeats from age 71 to age 75 when a third round of treatment is administered.

As the upper range of a subject's volume estimates reaches a threshold (for example 40 cc or 50 cc), the methods and systems herein can provide courses of medical action including: a prostate volume measurement (such as ultrasound or MRI imaging) to accurately estimate prostate size and calibrate PSA trends and digital rectal exam estimates of volume, or prostatitis analysis.

In an example, the methods and systems herein are used to monitor treatment effectiveness and monitor prostate volume growth after medical treatment has shrunk the prostate. For example, one year treatment with a combination of medicines can reduce prostate size by 25% for a typical man. If the prostate is treated early at moderate size, it may be possible to stop treatment after a year and then monitor often slow prostate growth for five years or more until the volume estimate indicates another year of treatment. Serious BPH symptoms may be avoided during this repeating cycle of treatment and monitoring.

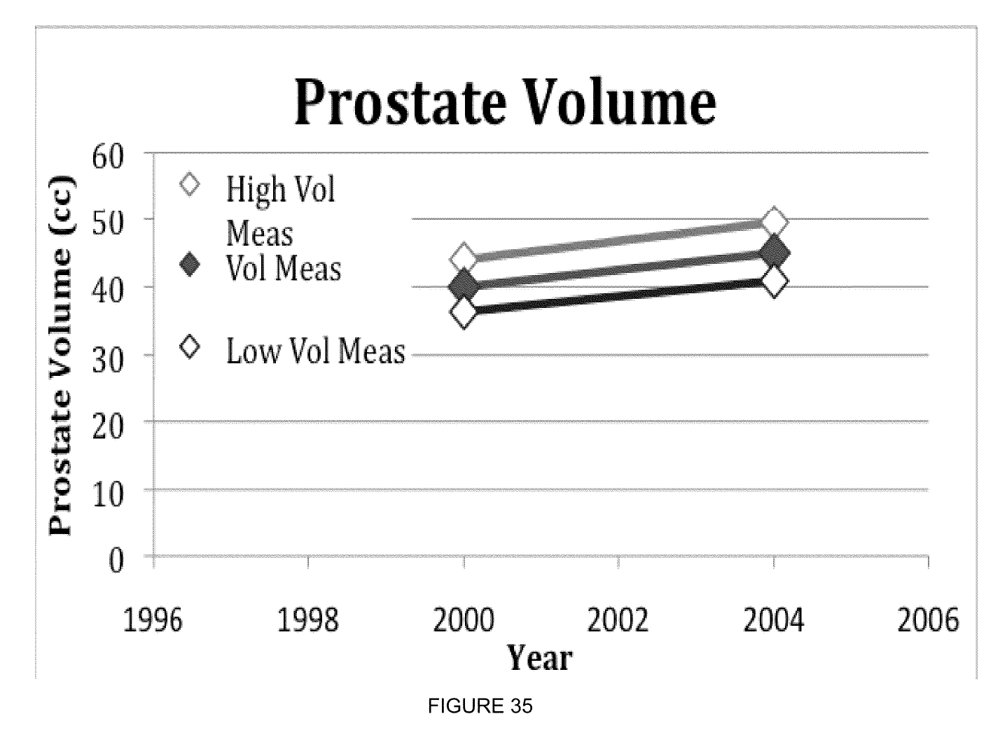

FIG. 35 demonstrates an exemplary method and system of the invention. Volume measurements are shown by diamonds. Two or more measurements are connected by a line. Volume measurements are not exact. The top diamond(s) with no center shows the high range(s) of the actual volume, and the bottom diamond(s) with no center shows the low range(s).

In some instances, prostate volume measurements inform a man about the size and growth rate of a prostate, and can be used to estimate the impact of the prostate's size and growth rate on the amount of PSA and Free PSA that the prostate produces over time. In many instances, the larger a prostate, the more PSA and Free PSA it produces. Slow volume growth over many years can lead to a large prostate that produces more PSA than a smaller prostate.

In an example, prostate volume estimates based on PSA trends give a sense of a subject's prostate size and how fast it might be growing. These trends can give medical personnel an early indication of BPH that might be treated before symptoms become serious. The trends can be based on a combination of a subject's PSA trends and one or more prostate volume measurements, if available.

Methods herein describing dynamic screening estimate prostate volume based on a subject's PSA trends and low, median and high PSA densities based on population data, if no volume measurements are available.

In an example, a method comprises obtaining a PSA trend and dividing the values along that trend by an estimate of the median PSA density to estimate a prostate volume trend. In a similar way, it divides the PSA trend values by a low value for PSA density to calculate the high estimate of the volume trend. Low PSA density is an estimate of the low tenth percentile of PSA density. In some instances, a method herein comprises dividing the PSA trend values by a high value for PSA density to calculate the low estimate of the volume trend. High PSA density is an estimate of the high tenth percentile of PSA density for men with BPH but not prostatitis.

When there is one existing volume measurement, a method or system herein can comprise using the volume measurement and verifying the volume estimate. Then it calculates the value of a PSA trend at the time of the volume measurement and divides this value by the volume measurement to estimate the PSA density. The method or system then divides the values along the PSA trend by this estimate of the PSA density to make an informed best estimate of the prostate volume trend. In some instances, there may be 10% or more variation in prostate volume measurements using ultrasound images, therefore visual output from the system may show a high volume trend that is 10% above the best estimate through the actual volume measurement and a low volume trend that is 10% below the best estimate.

In an example with two or more volume measurements, the methods and systems described herein comprise using the volume measurements and verifying the volume trend estimates. Next it calculates the value of the PSA trend at the time of each volume measurement. The system or method can divide this value by the volume measurement to estimate the PSA density at that time. It then divides the values along the PSA trend prior to the first measurement by the estimate of the PSA density using the first measurement to make an informed estimate of the prostate volume trend prior to the first measurement. It goes through a similar process after the last volume measurement using PSA density calculated based on the last measurement. To estimate prostate volume between measurements, the system uses a time weighted PSA density and the intervening PSA trend. At the early measurement, the system uses 100% of the PSA density calculated using the early measurement. At the late measurement, a method herein uses 100% of the PSA density calculated using the late measurement. Halfway between measurements in time, the system uses 50% of the PSA density based on the early measurement and 50% of the PSA density based on the late measurement. There may be 10% or more variation in prostate volume measurements using ultrasound images. A visual output from the system can show a high volume trend that is 10% above the best estimate through the actual volume measurement and a low volume trend that is 10% below the best estimate.

In another example, prostate volume estimates based on digital rectal exams (DREs) give a sense of the prostate size and how fast it might be growing. These trends can give a man early indications of BPH that might be treated before symptoms become serious. The trends can be based on a combination of a subject's PSA and DREs and one or more prostate volume measurements, if available.

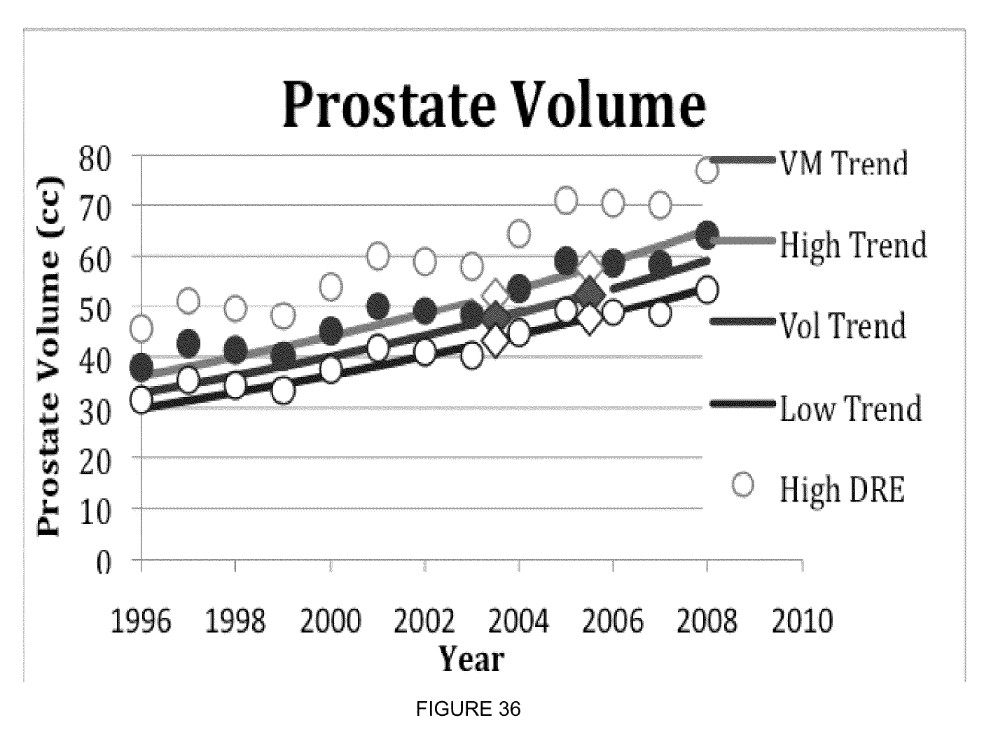

In some instances, DRE bias and accuracy can be adjusted for by the system and method herein. For example, a system or method herein can estimate the prostate volume trends based on DRE volume estimates, if no volume measurements are available. In another example, if available, one or more volume measurements can be used to verify or examine the estimates of prostate volume using DREs. Volume measurements are shown in FIG. 36 using solid lines connecting volume measurements.

In other embodiments herein, estimates of prostate volume trends can provide clues that chronic prostatitis is present and increasing or that DRE estimates are increasing faster than prostate volume is increasing. In an example, BPH causes prostates to grow slowly: 3% growth per year is relatively high, 4% is unusually high, 5% is rare and 6% or more is extremely rare. In this example, the faster the PSA growth rate rises above 3% per year the more likely chronic prostatitis is a cause of increasing PSA. The faster the DRE growth rate rises above 3%, volume estimation method can be adjusted.

In some instances, estimates of PSA density trends can provide additional clues that chronic prostatitis is present and increasing or that DRE estimates of volume after calibrations are still biased.

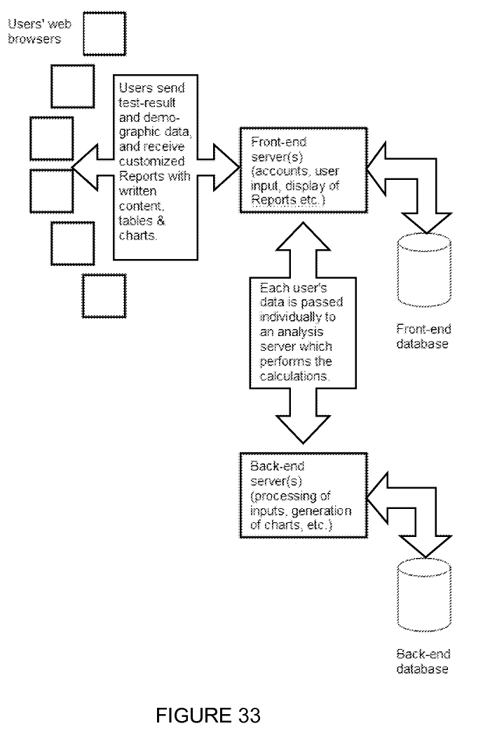

In some instances, PSA values may need to equilibrate after treatment (for example, falling to a new lower level) or after the stop of treatment (for example, increasing to a new higher level). After this has occurred, PSA can then provide information about prostate volume, for example with a new equilibrium PSA density. Often PSA equilibrates after treatment within a few weeks or months. This example is demonstrated in FIG. 37.

### Prostatitis

In another aspect, a detection process system is disclosed for the detection of prostatitis. In some embodiments, the detection process system can be incorporated into a system or method for calculating the probability of progressing prostate cancer. In some instances, a detection process system provides doctors, and men, decisions on medical actions for detecting and/or treating prostatitis. In some instances, a detection process system can interpret results in order to estimate the probability of prostatitis.

In some instances, an elevated PSA may suggest the possibility of prostatitis if there is an anomalous PSA increase in a trend or series of PSA measurements of a man over time. The onset or increase in severity of inflammation or infection of the prostate can cause an increase in PSA that is inconsistent with a trend. The anomalous value can be used in a method disclosed herein to determine the probability of the occurrence of prostatitis. In some instances, a method or system disclosed herein may identify the probable cause of the anomalous PSA value with more confidence.

In other instances, chronic inflammation or infection that is increasing in severity can cause an increasing PSA trend. A probability of prostatitis can be calculated from the PSA trend and the methods and systems can be used to identify the probable cause of the increasing PSA trend with more confidence. Prostatitis is an inflammation and/or infection of the prostate gland in men.

In an embodiment, a system comprises: a user interface that can collect data and send it to a system and can receive content from the system and display. A system can also comprise a system that creates custom content and graphs system incorporated into the system. A quantitative analysis system may receive data and instructions, perform quantitative analysis, send data and analysis results, and instructions. The systems may comprise Bayesian network interface and Bayesian network that performs Bayesian network calculations based on instructions and data from the system.

The National Institute of Diabetes and Digestive and Kidney Diseases (NIDDK) devised a new classification system in 1999, comprising four categories of prostatitis: Acute prostatitis is a bacterial infection of the prostate gland that requires urgent medical treatment; Chronic bacterial prostatitis is a relatively rare condition that usually presents as intermittent urinary tract infections; Chronic Prostatitis/Chronic Pelvic Pain Syndrome (CP/CPPS), is also known as chronic nonbacterial prostatitis; and Asymptomatic inflammatory prostatitis patients have no history of genitourinary pain complaints, but leukocytosis is noted, usually during evaluation for other conditions.

Exemplary treatments include antibiotics as a first line of treatment in acute prostatitis. Antibiotics usually resolve acute prostatitis infections in a very short time. Appropriate antibiotics should be used, based on the microbe causing the infection. In some instances, patients in urinary retention are best managed with a suprapubic catheter or intermittent catheterization.

In many instances of chronic bacterial prostatitis, there are bacteria in the prostate but often no symptoms. Often, the prostate infection is diagnosed by culturing urine as well as prostate fluid (expressed prostatic secretions or EPS) which can be obtained by the doctor doing a rectal exam and putting pressure on the prostate. PSA levels may be elevated, although there is no malignancy. Treatment of chronic bacterial prostatitis can include prolonged courses (4-8 weeks) of antibiotics and/or alpha blockers (such as tamsulosin (Flomax), alfuzosin).

Chronic prostatitis/chronic pelvic pain syndrome (CP/CPPS) is characterized by pelvic or perineal pain without evidence of urinary tract infection, lasting longer than 3 months. For chronic nonbacterial prostatitis (Cat III), a treatment called the “Stanford Protocol”, has recently been published. This is a combination of medication (using tricyclic antidepressants and benzodiazepines), psychological therapy (paradoxical relaxation, an advancement and adaptation, specifically for pelvic pain, of a type of progressive relaxation technique), and physical therapy (trigger point release therapy on pelvic floor and abdominal muscles, and also yoga-type exercises with the aim of relaxing pelvic floor and abdominal muscles). While these studies are encouraging, definitive proof of efficacy would require a randomized, sham controlled, blinded study, which is not as easy to do with physical therapy as with drug therapy. There is a substantial list of medications used to treat this disorder, such as Alpha blockers (for example, tamsulosin, alfuzosin), Quercetin, and pollen extract (for example, Cernilton). In some embodiments, the methods of treatments herein for prostatitis described herein can be a course of medical action carried out as part of a method described herein.

Asymptomatic inflammatory prostatitis is a symptomless microscopic condition of the prostate gland. It should be distinguished from other forms of prostatitis such as chronic bacterial prostatitis, acute bacterial prostatitis and chronic pelvic pain syndrome (CPPS). These patients have no history of genitourinary pain complaints, but leukocytosis is noted, usually during evaluation for other conditions. Medical actions can be performed to diagnose through tests of semen, EPS or prostate tissue that reveal inflammation in the absence of symptoms.

As described herein a system or method can calculate consistent trends from all consistent test results of a prostate biomarker. Trends are estimated using exponential curves when there is enough data. Velocities (annual rates of change) are calculated for both PSA and Free PSA trends. Ratios of trend values and velocities are calculated (fPSA % and fPSAV %). Trend values and recent test results are passed to other components. The probabilities of prostatitis can be calculated for inflammation and infection based on prior probabilities, trends and a limited amount of other data. Probabilities of inflammation and infection tend to be much higher for inconsistent elevated last PSA test caused by a temporary condition than for a slowly increasing PSA trend caused by a chronic condition. The posterior probabilities that are calculated by the system can be sent to a Bayesian network. This component can calculate changes in PSA, fPSA and their ratio for changes from previous test pairs and from the trends. The changes can be used to calculate probabilities of conditions indicated by the changes in PSA. The drop in PSA and Free PSA, if any, from a previous test pair or trend after treatment by anti-inflammatory meds and/or antibiotics may be of particular interest.

In an embodiment, a component translates anti-inflammatory or antibiotic medication types, doses and durations into a one dimensional effectiveness output measure. A one dimensional output is used to simplify a Bayesian network and specification of parameters for it. Effectiveness can be defined as the ability to reduce the effects of inflammation. A wider variety of medications (concurrent or sequential) may be more likely to reduce inflammation than a smaller number of medications. When appropriate for a particular type of inflammation, a higher dose of a medication for a longer duration can be more effective, albeit with diminishing returns to both dimensions. Combinations of medications might have the following potential effectiveness as shown in Table 1

TABLE 1
Potential Effectiveness of Medications (PE)

| Medications | | | Potential |
|---|---|---|---|
| Med A | Med B | Med C | Effectiveness |
| A | B | C | 99% |
| A | B | | 75% |
| A | | C | 70% |
| A | | | 40% |
| | B | C | 65% |
| | B | | 35% |
| | | C | 30% |

For a given medication, combinations of dose and duration of treatment might have the following actual effectiveness as shown in Table 2.

TABLE 2
Medication Effectiveness (ME)

| | | Duration (months) | | |
|---|---|---|---|---|
| | | 1 | 2 | 3 |
| Dose | High | 80% | 95% | 100% |
| | Medium | 75% | 90% | 95% |
| | Low | 60% | 75% | 80% |

Total effectiveness (TE) of a one medicine or a combination of medicines (concurrent or sequential) may be:

TE(a)=MEa(d,m)*PE(a)

TE(b)=MEb(d,m)*PE(b)

TE(c)=MEc(d,m)*PE(c)

TE(ab)=MEao(d,m)*PE(ao)+MEa/b(d,m)*PE(a/b)+MEbo(d,m)*PE(bo)

TE(ac)=MEao(d,m)*PE(ao)+MEa/c(d,m)*PE(a/c)+MEco(d,m)*PE(co)

TE(bc)=MEbo(d,m)*PE(bo)+MEb/c(d,m)*PE(b/c)+MEco(d,m)*PE(co)

TE(abc)=MEao(d,m)*PE(ao)+MEbo(d,m)*PE(bo)+MEco(d,m)*PE(co)+MEa/b(d,m)*PE(a/b)+MEa/c(d,m)*PE(a/c)+MEb/c(d,m)*PE(b/c)+MEa/b/c(d,m)*PE(a/b/c)

Each additional medication adds an incremental amount of potential effectiveness and that incremental amount is then weighted by the medication effectiveness for that incremental med. For example, medication A alone offers a total effectiveness of:

TE(a)=MEa(d,m)*PE(a)

Similarly, medication B alone offers a total effectiveness of:

TE(b)=MEb(d,m)*PE(b)

A combination of med A and B offers a total effectiveness of:

TE(ab)=MEao(d,m)*PE(ao)+MEa/b(d,m)*PE(a/b)+MEbo(d,m)*PE(bo)

Where ao (A Only) reflects the impact of medication A on conditions not affected by medication B, bo (B Only) reflects the impact of medication B on conditions not affected by medication A, and a/b (A and B) reflects the impact of medications A and B on conditions affected by both medications.

As a first approximation, it can be set:

MEao(d,m)=MEa(d,m)

MEbo(d,m)=MEb(d,m)

It can also be solved for the potential effectiveness of A and B only when used in combination:

PE(ao)=PE(ab)−PE(b)

PE(bo)=PE(ab)−PE(a)

In addition, it can be solved for the potential effectiveness of medication A and medication B for conditions they overlap on:

PE(a/b)=PE(ab)−PE(ao)−PE(bo)=PE(ab)−PE(ab)+PE(b)−PE(ab)+PE(a)=PE(a)+PE(b)−PE(ab)

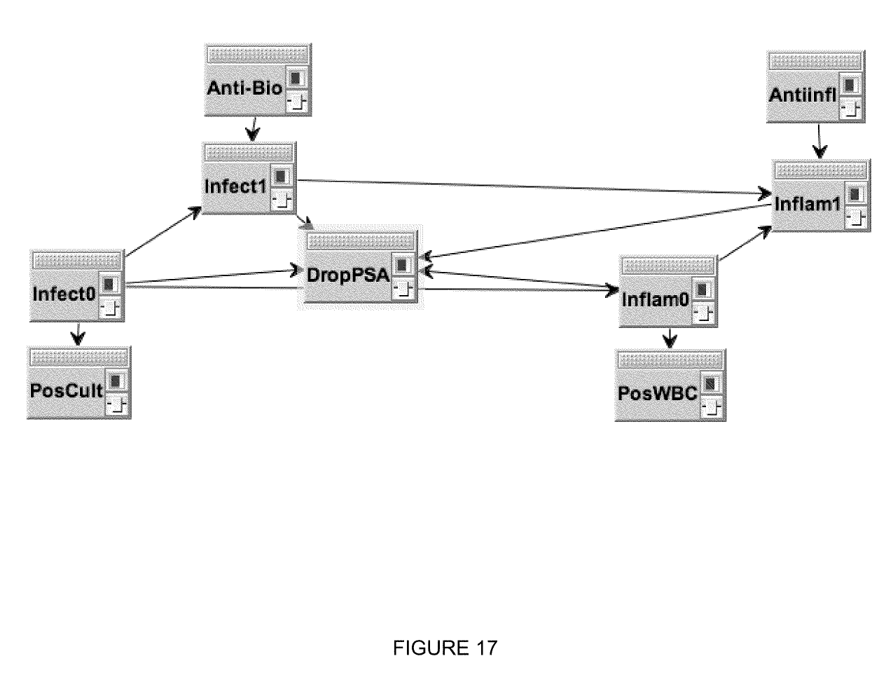

A simplified version of the Bayesian network as described herein for the prostatitis detection process is in FIG. 17.

In FIG. 17, Infections 0 refers to the initial infection state prior to treatment. For example purposes, priors are: 10% True, 90% False. PosCult refers to a positive culture from growing bacteria from prostate secretions. A positive culture indicates the presence of bacterial infection. It is influenced by infections 0. Anti-bio refers to antibiotics can reduce the strength of infections. For example, initial priors are: 50% True, 50% False for the anti-bio. Infections 1 in FIG. 17 refers to the subsequent infection state after treatment. Inflammations 0 refers to the initial inflammation state prior to treatment. A count of white blood cells in prostate secretions can help identify inflammation of the prostate. The higher the WBCC above normal the more likely inflammation is present in the prostate, this test is shown in the Bayesian network in FIG. 17 by PosWBC. Anti-inflammatory medications (AntiInfl) can reduce the strength of inflammations. These anti-inflammatory medications are action variables. For example, initial priors are: 50% True, 50% False. Inflammations 1 refers to the initial inflammation state subsequent to treatment. As shown in FIG. 17, Drop in PSA is an input for example percentage drops such as 80% and greater, 60%-80%, 40%-60%, 30%-40%, 20%-30%, 10%-20%, 0%-10%, negative 10%-0%, negative 20%-negative 10%, and negative 30% and greater

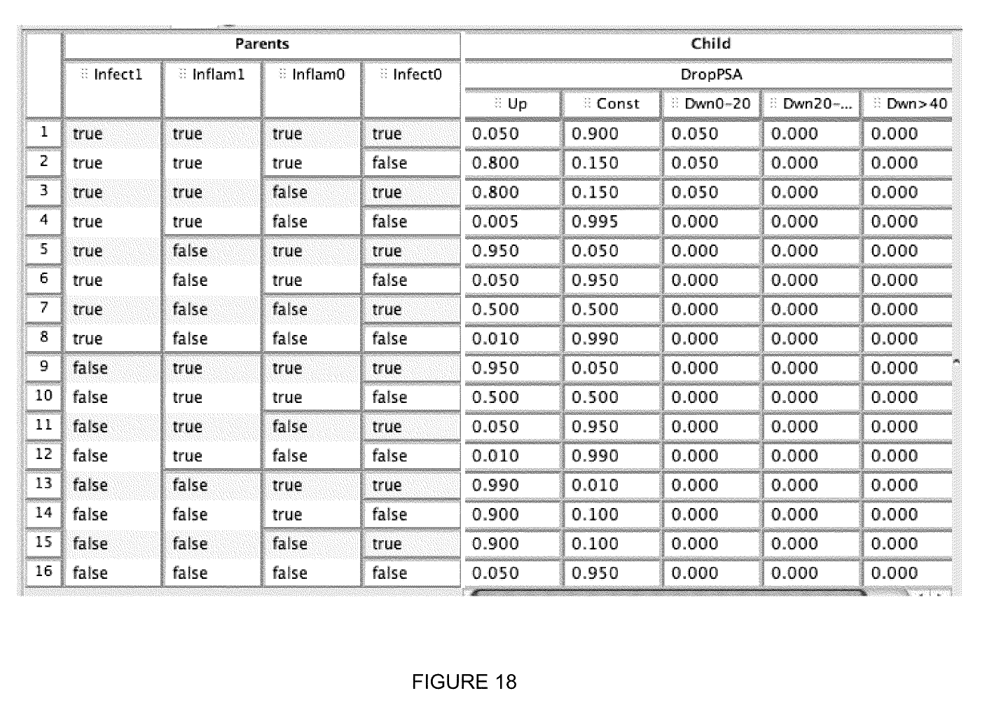

FIG. 18 shows a Bayesian network. The figure shows that the larger the percentage drop in PSA from the previous test to the most recent test the more likely prostatitis was a cause of the elevated previous test.

Currently, doctors use crude rules of thumb for treatment of prostatitis caused by inflammation and infection with little or no quantitative measurement and feedback to personalize treatment or optimize their rules of thumb over time. PSA and Free PSA provide substantial amounts of information about the course of prostatitis if cancer is not progressing to an advanced stage and prostate volume is measured to take the effects of BPH into account.

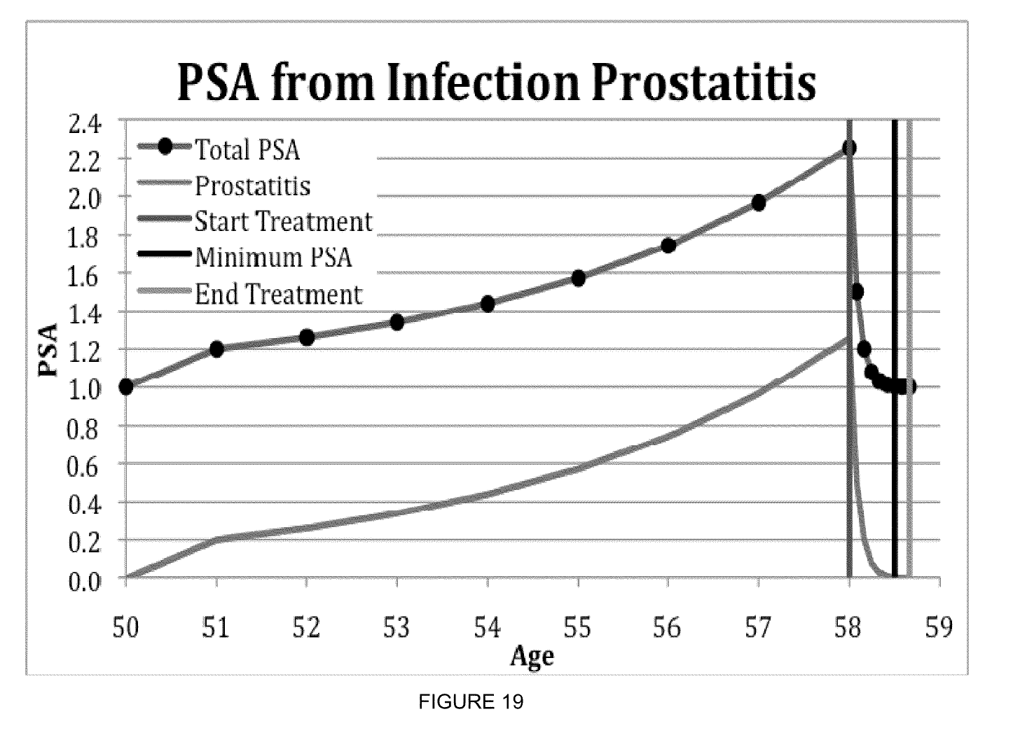

In an example, consider a man with a healthy prostate that produces 1.0 of measured PSA at age fifty, shown by the dot at the left of FIG. 19. Soon after, a mild infection starts growing slowly in his prostate, shown by the total PSA curve with dots for measurements and the implied infection PSA curve. By age 58 in FIG. 19, the infection is producing a little over 1.2 of PSA, and the man's total measured PSA is a little over 2.2. At that point, his doctor initiates a course of antibiotic treatment, shown by the vertical line.

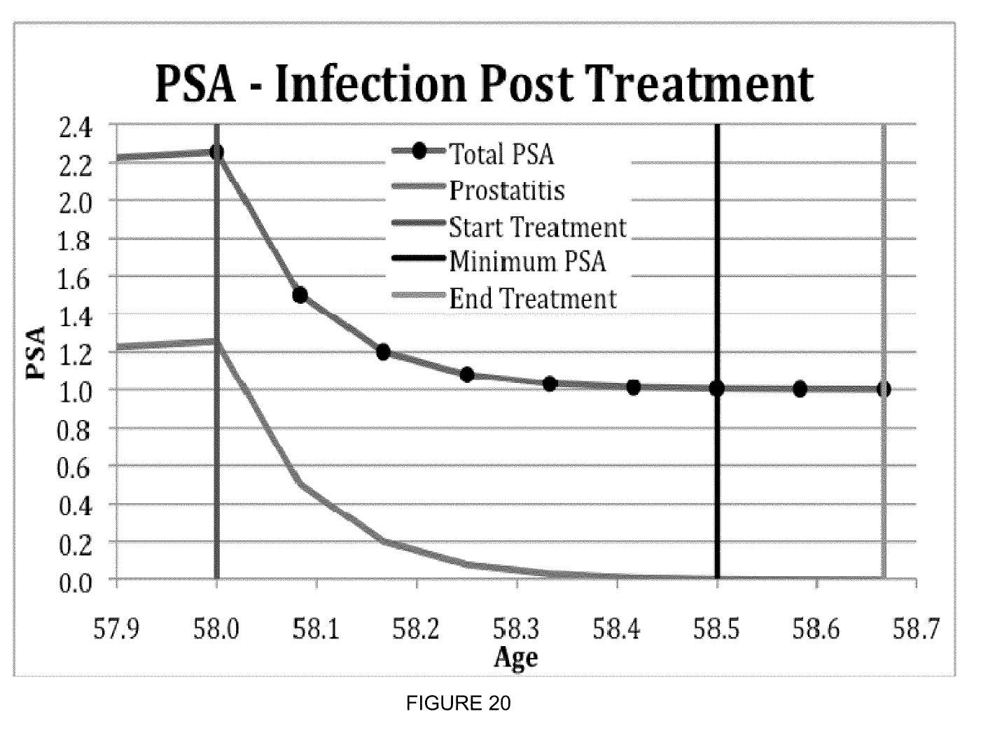

The vertical line in FIG. 20 shows the start of a course of antibiotic treatment using a standard dose of a typical antibiotic. The curve shows total PSA, and the dots show PSA measurements. The curve shows implied PSA from the infection. In some instances, treatment of prostate infections can be a long process because antibiotics do not penetrate the prostate easily or thoroughly and some bacteria can escape treatment for many months or longer. Often, medical personnel have no effective way of determining when treatment can be stopped with confidence that the infection is entirely cured and will not flare up again. In some instances, symptoms provide little help because they often disappear within the first days or weeks of treatment. The methods described herein of dynamic treatment monitoring using PSA can tell the doctor when PSA reaches a minimum, shown by the vertical line. The doctor can then wait an additional period to allow the antibiotic to kill all the bacteria before ending treatment, shown by the bright vertical line in FIG. 20.

Detailed records of treatment end points (measured as weeks after the minimum PSA point) and frequency of and time to recurrence for many patients can allow optimization of end point rules. Treatment length can be increased until short term recurrence rates are driven to an appropriately low level.

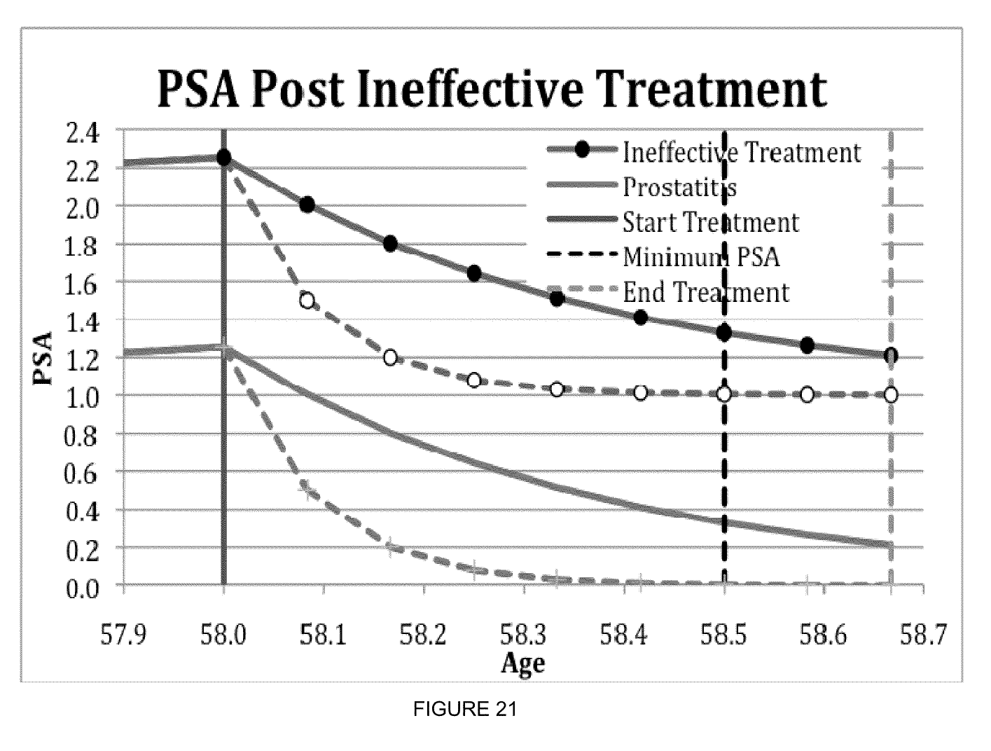

Prostates can be infected by a range of bacteria. Most antibiotics is effective against some bacteria and ineffective against others. In some embodiments, the methods herein provide early detection of ineffective treatment allows the user to switch to another antibiotic and minimize total treatment time, cost and side effects. Dynamic monitoring methods and systems as described herein using PSA tests can help the doctor detect ineffective treatment earlier than current practice (which may not identify ineffective treatment until symptoms recur). The dotted and curves in FIG. 21 show PSA trends for the effective treatment. The solid and curves show PSA trends for ineffective treatment. One PSA test after treatment and an expectation for effective treatment is enough to identify ineffective treatment very early. A second PSA test can confirm treatment ineffectiveness.

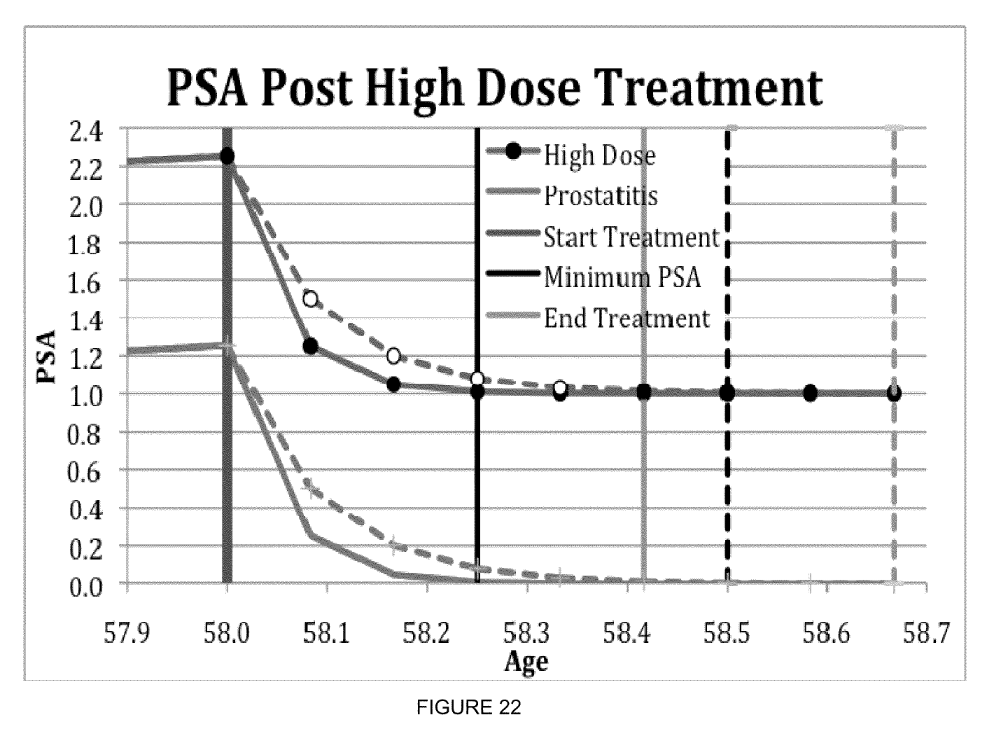

The wide vertical line in FIG. 22 shows the start of a course of antibiotic treatment using a high dose of a typical antibiotic. The solid curve shows total PSA for high dose treatment, and the dotted curve shows standard dose. The solid curve shows implied PSA from the infection for high dose treatment, and the dotted curve shows standard dose. The treatment period is shortened substantially from the bright dashed vertical line for standard dose to the solid vertical line for high dose. Dynamic monitoring herein allows the doctor to shorten the course of treatment using high dose antibiotics without compromising treatment effectiveness.

Detailed records of antibiotic dose, treatment end points (measured as weeks after the minimum PSA point) and frequency of and time to recurrence for many patients will allow optimization of dose and end point rules. Treatment length can be increased until short term recurrence rates are driven to an appropriately low level. Dose can be varied to provide optimal combinations of dose and treatment length for the doctor and patient to choose from.

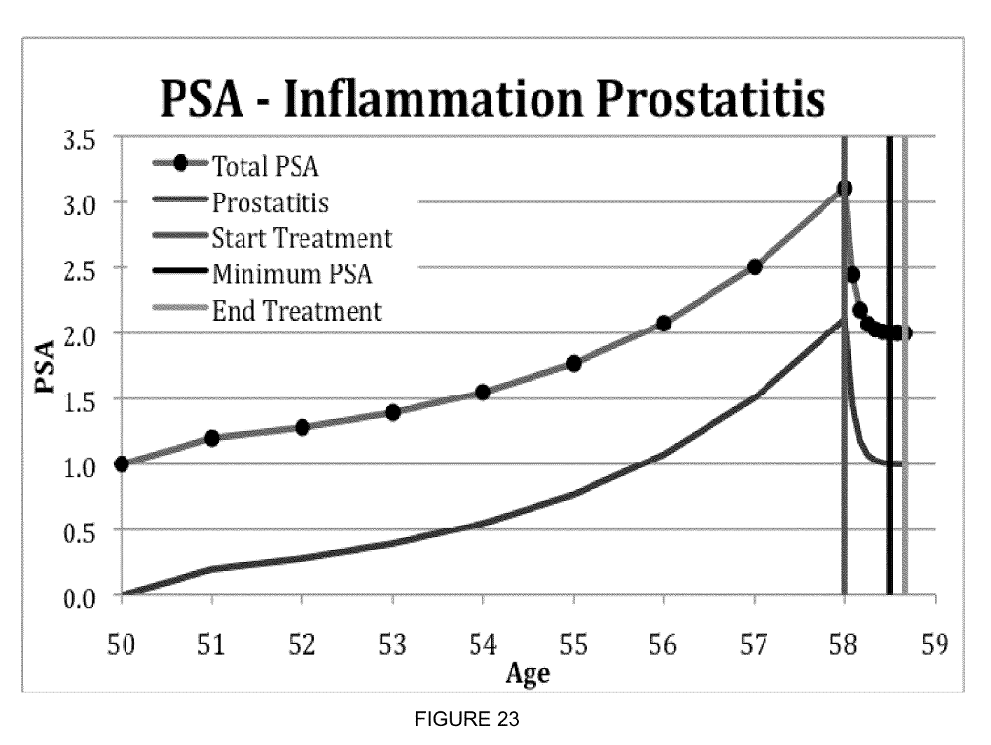

In another example, a man with a healthy prostate produces 1.0 of measured PSA at age fifty, shown by the dot at the left of FIG. 23. Soon after, mild inflammation starts to affect his prostate with growing severity, shown by the total PSA curve with dots for measurements and the implied inflammation PSA curve. By age 58 in FIG. 23, the inflammation is producing about 2.1 of PSA, and the man's total measured PSA is a little over 3.1. At that point, a method includes initiating a course of anti-inflammatory medication, shown by the vertical line.

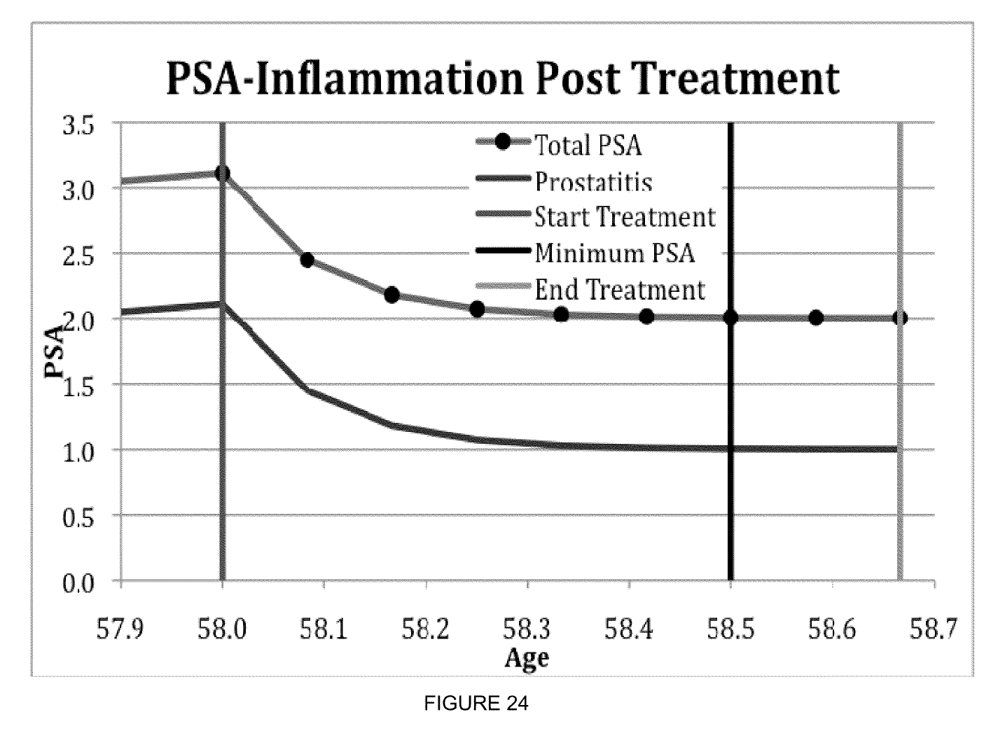

The vertical line in FIG. 24 shows the start of a course of treatment using a standard dose of a typical anti-inflammatory medication. The curve shows total PSA, and the dots show PSA measurements. The curve shows implied PSA from the inflammation. Treatment of prostate inflammations can be a long process and may never result in a cure. Often, doctors have no effective way of determining when treatment can be stopped with confidence that the inflammation has been minimized by a given medication. Symptoms may provide little help because they often disappear within the first days or weeks of treatment. Dynamic treatment monitoring as described herein using PSA can tell the doctor when PSA reaches a minimum, shown by the vertical line. The doctor can then wait an additional period to allow the medication to do all it can before ending treatment, shown by the bright vertical line. In this example, inflammation indicated by PSA has been reduced but not eliminated with no further reduction likely. At this point, the doctor can change medication in hopes of further reduction in inflammation, stop medication and monitor PSA or continue medication (perhaps at a lower dose) and monitor PSA.

Dynamic monitoring of PSA after treatment of cured prostatitis can give the doctor early warning of recurrence. Repeated recurrences can give the doctor data that will help optimize subsequent treatment.

In some instances, methods and systems disclosed herein can provide early detection and treatment of prostatitis, and present probabilities of temporary prostate conditions and chronic prostatitis.

Medical researchers are increasingly contemplating the benefits of early detection and treatment of prostatitis. Often, the primary focus has been on improving PSA screening for prostate cancer. However, there is increasing attention to early treatment of prostatitis. Non-life-threatening prostatitis is a prostate condition that can be caused by inflammation and/or infection. It can cause a variety of symptoms, including a frequent and urgent need to urinate and pain or burning when urinating, often accompanied by pelvic, groin or low back pain. In some instances, prostatitis may be the first cause to consider if a PSA biomarker value is increasing. In an example, a sharp increase in PSA normally signals a temporary condition (acute prostatitis or irritation). As described herein, probabilities of inflammation, infection and irritation are calculated for sharp increases in PSA. In some instances, steady increases typically indicate long-term inflammation or infection (chronic prostatitis). By comparing trends of a subject with those of men who have developed these conditions, the methods and systems described herein can calculate the probability of experiencing chronic prostatitis caused by inflammation or infection. In an embodiment, the ratio of changes in free PSA to PSA helps distinguish between inflammation (high ratios) and infection (low ratios).

As the probability of inflammation and/or infection increases, the methods and systems disclosed herein may suggest a course of medical action, such as a culture and/or white blood cell count of prostate secretions and treatment with anti-inflammatory therapeutics and/or antibiotics.

In some instances, early detection of prostatitis allows the option of treatment before symptoms appear or become serious, which may avoid potential damage from undetected chronic prostatitis. For example, during treatment, a rapidly falling PSA suggests effective treatment that can be relatively short. In contrast, PSA that does not decrease or decreases slowly suggests ineffective treatment and that treatment time or treatment may need adjustment.

In an embodiment, the methods and systems herein can estimate the probability that a sharp increase in PSA corresponds to temporary inflammation, infection, or irritation by analyzing how closely the PSA data reflects changes typical for each of these conditions. For most men, a temporary inflammation of the prostate usually causes a sharp increase in PSA without a sharp drop in the ratio of Free PSA to PSA, a temporary infection of the prostate usually causes a sharp increase in PSA and a sharp drop in the ratio of Free PSA to PSA, and temporary irritation of the prostate usually causes a sharp but small increase in PSA without a sharp drop in the ratio of Free PSA to PSA.

In an example, there may be three indicators of chronic prostatitis including the PSA growth rate. Chronic prostatitis and an enlarged prostate caused by BPH may be the cause of elevated PSA trends. However, the PSA growth rate as described may help distinguish between them. In an example, prostate volume and the corresponding PSA grow very slowly for most men (in the 0% to 2% range, 3% is unusually high, 4% is rare, and 5% or more is very rare). The higher the PSA growth rate above 0% to 2% the more likely it is caused by increasing chronic prostatitis. In some cases a high PSA growth rate is an indication of progressing cancer.

In another example, a typical healthy prostate has a PSA density of around 0.04 (1.0 PSA and 25 cc volume). It is unusual to have a PSA density above a high range of 0.05 to 0.06 or more unless prostatitis is affecting the prostate. PSA density tends to decrease slowly as prostates enlarge. Therefore, PSA density growth rates greater than 3% to 5% or more per year can be indicators of increasing chronic prostatitis.

In yet another example, prostatitis can be the primary cause of substantial PSA test variability around the trend. PSA from volume growth tends to increase slowly and smoothly. The methods and systems herein can utilize a form of coefficient of variation based on percentage deviations from trend. In this example, variation from 0% to 10% is expected from normal random variation. Variation from 10% to 20% may raise suspicion of prostatitis. Variation from 20% to 30% may be caused by prostatitis.

PSA and free PSA (fPSA) may provide substantial amounts of information about the course of prostatitis (for example, if cancer is not progressing and prostate volume is measured to take the effects of BPH into account). Currently, many doctors use rules of thumb for treatment of prostatitis caused by inflammation and infection with little or no quantitative measurement and feedback.

Treatment of prostate infections can be a long process because antibiotics do not penetrate the prostate easily or thoroughly and some bacteria can escape treatment for many months or longer. Treatment of prostate inflammations can also be a long process and may never result in a cure. In some cases, doctors may have no effective way of determining when treatment can be stopped with confidence that the infection and/or inflammation is entirely cured and is unlikely to flare up again. Symptoms may not provide clues because they often disappear within the first days or weeks of treatment. In an embodiment, the systems and methods herein comprise monitoring using PSA and provide to the user or medical personnel when PSA reaches a threshold. Based on this feedback the course of medical action or medical treatment can be adjusted.

### Dynamic Screening of a Plurality of Prostate Medical Conditions

In an embodiment, first and second values are a first and second biomarker trend of prostate biomarker values over a period of time. A computer system can calculate each of said first plurality of posterior probabilities by relating: a prior probability of a prostatic medical condition; a probability of observing said first biomarker trend for an individual with said condition; and a probability of observing said first biomarker trend for an individual without said medical condition. A computer system can calculate each of said second plurality of posterior probabilities by relating: a prior probability of a prostatic medical condition, wherein said prior probability was calculated using subject information comprising said result of a course of medical action; a probability of observing said second biomarker trend for an individual with said condition; and a probability of observing said second biomarker trend for an individual without said medical condition.

In an embodiment, prostate medical conditions, for example, can be selected from the group consisting of the following: a plurality of types of prostatitis as described, prostate cancer, benign prostate hyperplasia, and no prostate disease. The prostate biomarker can be any prostate biomarker. In many instances, the biomarker is prostate specific antigen (PSA) or free PSA.

In an embodiment, a first or second course of medical action is delivering medical treatment to said subject, such as a medical treatment is selected from a group consisting of the following: a pharmaceutical, antibiotics, antiinflammatories, TURP, TUNA, TUMT, radical prostatectomy, other surgery, organ resection, and radiation therapy. In another embodiment, the first or second course of medical action comprises administration of medical tests or medical imaging of said subject or setting a specific time for delivering medical treatment or a biopsy or a consultation with a medical professional. The course of medical action can also comprise administration of medical tests, medical imaging of said subject, setting a specific time for delivering medical treatment, a biopsy, and/or consultation with a medical professional.

In yet another aspect, a method of delivering a probability that a subject has a medical condition to a user is disclosed comprising: calculating a plurality of posterior probabilities of the occurrence of a plurality of prostate medical conditions of a subject having a PSA value and an fPSA value, each at more than one time thereby having a PSA trend and an fPSA trend, wherein each of said plurality of posterior probabilities is calculated by relating: a prior probability of a prostate medical condition; and a probability of observing said PSA trend and said fPSA trend for an individual with said prostate medical condition; and a probability of observing said PSA trend and said fPSA trend for an individual without said prostate medical condition; and delivering said plurality of probabilities of said plurality of medical conditions to a user with an output device. In an embodiment, a method can further comprise: calculating a second plurality of posterior probabilities of the occurrence of said plurality of prostate medical conditions of a subject having a result of a course of medical action and having a new PSA value and a new fPSA value, each at more than one time thereby having a second PSA trend and a second fPSA trend, wherein each of said plurality of posterior probabilities is calculated by relating: a prior probability of a prostate medical condition, wherein said prior probability was calculated using subject information comprising said result of a course of medical action; and a probability of observing said second PSA trend and said second fPSA trend for an individual with said prostate medical condition; and a probability of observing said second PSA trend and said second fPSA trend for an individual without said prostate medical condition; and delivering said second plurality of probabilities of said plurality of medical conditions to the user with an output device.

A system to perform the Bayes calculation of the probability of a prostatic disease can be configured with the following components: 1) prior probabilities of disease at various stages of progression; 2) probability of the observation of various prostate biomarker trends conditional on no disease; and 3) probability of the observation of various prostate biomarker trends conditional on various stages of disease.

For example, consider a man concerned about prostate cancer with a series of PSA and free PSA biomarker results from blood tests. Trends can be estimated for each biomarker and analyzed using methods previously disclosed. For example, trend PSA velocity is the annual rate of change in trend PSA; trend free PSA % is trend free PSA divided by trend PSA; and trend free PSA velocity % is trend free PSA velocity divided by trend PSA velocity. The results can be as in Table

TABLE 3
Example of values for biomarker trends.

| trend | Value | Standard Deviation |
|---|---|---|
| trend PSA | 3.0 | 0.4 |
| trend PSA velocity | 0.40 | 0.20 |
| trend free PSA % | 17.0% | 2.0% |
| trend fPSA velocity % | 6.0% | 3.0% |

Other information about the man may be available, including, age, measurement of prostate volume in some cases, and other factors that may affect the conditional probabilities.

Typically, no highly specific conditional distributions can be estimated directly from available population data. In an aspect, a disclosed method calculates the needed personalized probabilities.

The dynamic screening system as described herein can recognize the false alarms caused by infection and other temporary conditions, provide a calming perspective, suggest new PSA and free PSA tests after the infection or condition has passed, and analyze the results of new tests.

Embodiments herein extend the capabilities of dynamic screening. The capabilities relate to multiple benign conditions as well as progressing cancer. Included are descriptions covering temporary benign conditions, long-term conditions, both benign and cancer, and tuning distributions using long-term conditions as the example.

PSA and free PSA tests can have results that are greater than or smaller than their predicted trend values. In some instances, a method comprises labelling them anomalous and excludes them from subsequent trend estimation if their deviations, including the ratio free PSA to PSA, exceed certain tolerance ranges. Anomalous results with PSA values substantially below the trend are rare and may be caused by a variety of situations, including test error or test recording error. Anomalous results with PSA values substantially above the trend are more frequent and may be caused by one or more benign conditions. In some instances, a method estimates the probability of these benign conditions using Bayesian processes.

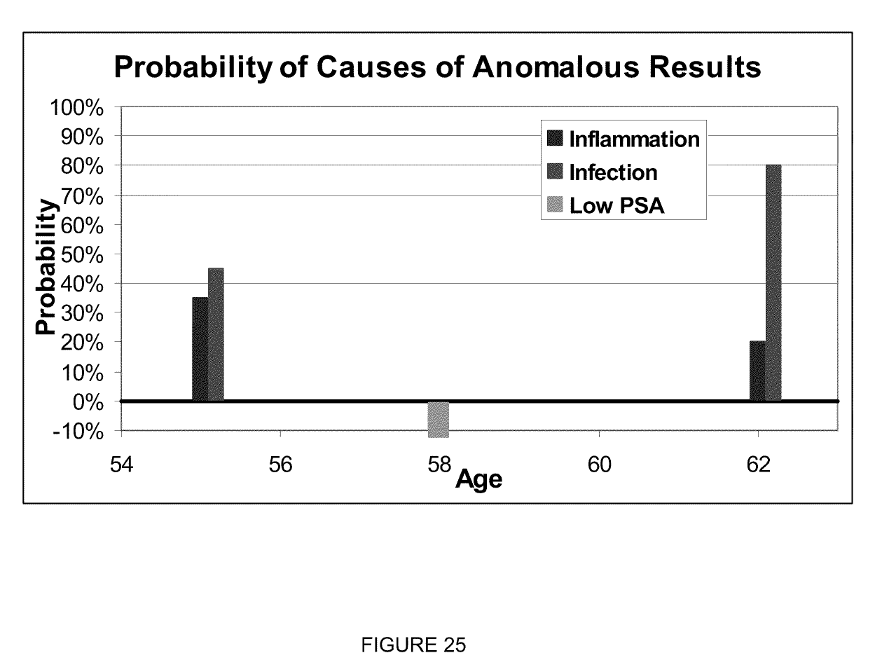

In an embodiment, capabilities are added to dynamic screening to allow the calculation of the probability of benign prostate conditions. Two exemplary conditions include, but are not limited to, inflammation prostatitis and infection prostatitis along with a category for other temporary conditions. FIG. 25 shows the probability of each of these two temporary benign conditions for anomalous test results over time for a man. These probabilities can be used to inform decisions about imaging, testing and treatment of possible conditions. Anomalous test results with PSA values below the trend are indicated by gray bars below the horizontal axis.

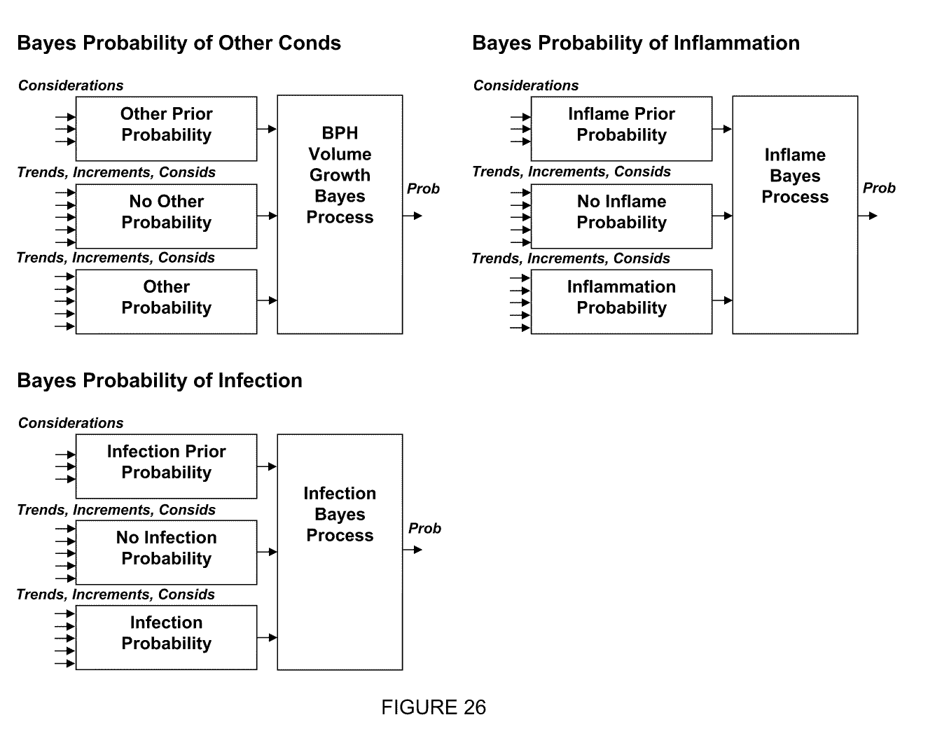

Three similar Bayes processes can be used to calculate the probability of the prostate conditions: inflammation prostatitis, infection prostatitis and other temporary conditions. The process for calculating the probability of progressing cancer has been disclosed previously. In an embodiment, the Bayes process uses three elements: the prior probability of the condition, the probability of the observed trend values and the incremental change from them conditional on all conditions and the probability of the observed trend values and the incremental change from them conditional on the absence of the condition but with all other conditions possible. Prior probabilities may be a function of age, race, genetics, demographics, past experience with the conditions and other considerations as shown in FIG. 26.

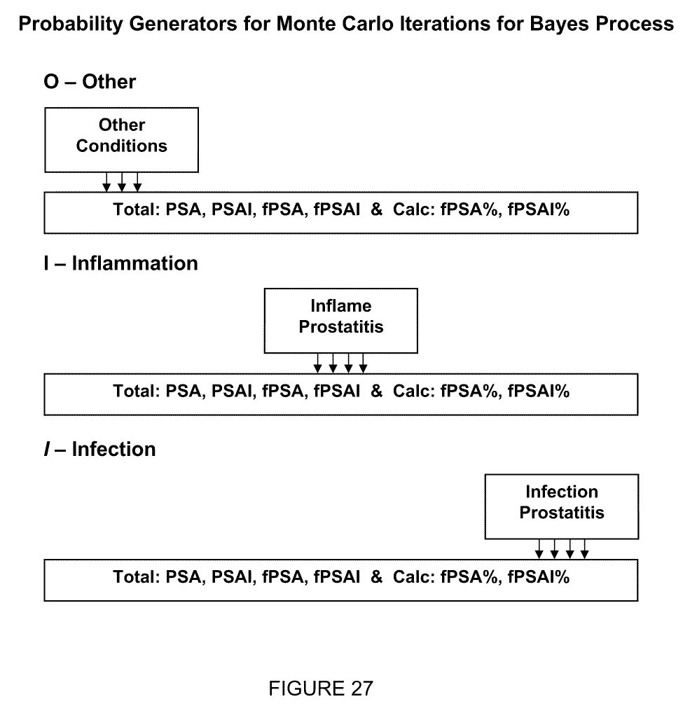

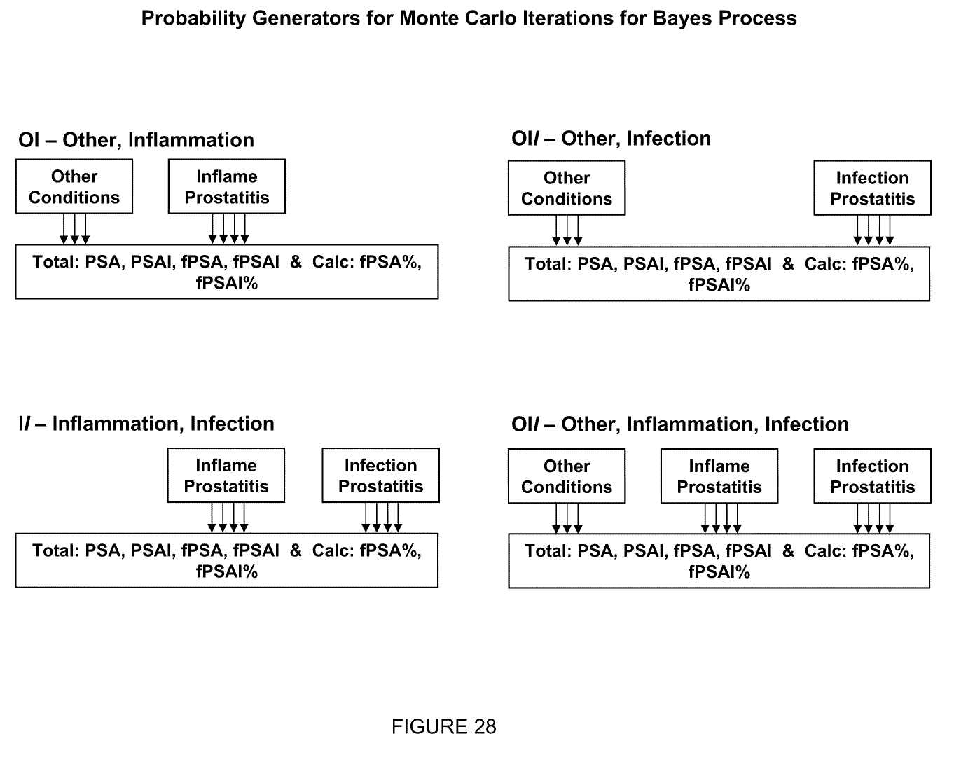

For example, the dynamic screening probability generator for temporary prostate conditions consolidates output from three separate probability generators: inflammation prostatitis, infection prostatitis and other temporary conditions as shown in FIG. 27 and FIG. 28. Total values are stored from iterations of the Monte Carlo process for four variables: PSA and PSA increment from the trend, and free PSA and free PSA increment from the trend. Ratios are calculated for free PSA % (=free PSA/PSA) and free PSA Increment % (=free PSA increment/PSA increment). Other probability generators are similar with one module removed. For example, the other condition generator starts with the all temporary prostate conditions generator and removes the other conditions generator.

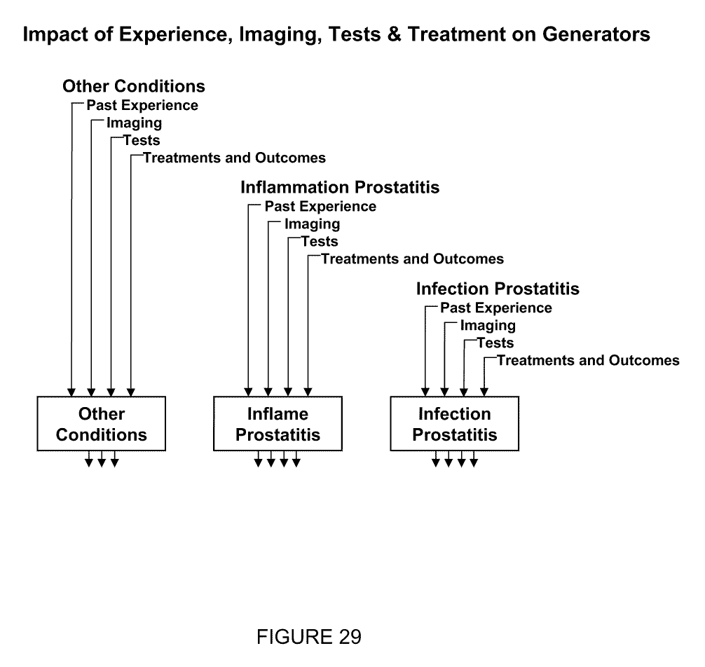

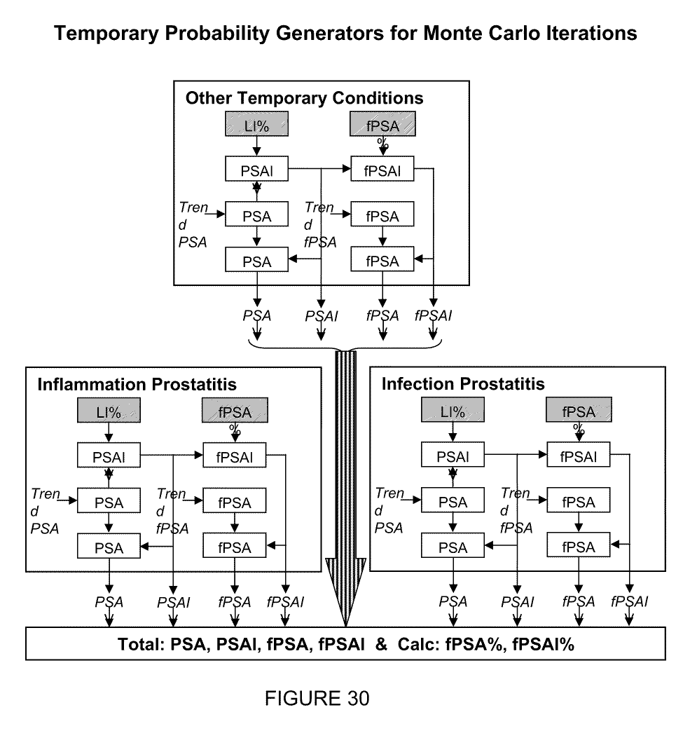

The probability distributions of each prostate condition can be affected by past medical experience with the conditions, and the results of imaging, tests, treatment and other medical procedures as shown in FIG. 29. For example, prostatic secretions can be cultured for bacterial infections. The results can affect the probability distributions produced by the infection prostatitis module. In a similar way, treatment with antibiotics can affect PSA levels. The outcome can affect the distributions produced by the infection prostatitis module. For example, a negative bacterial culture and no impact from antibiotic treatment may reduce the probability of infection prostatitis and increase the probability of inflammation and the probability of other conditions. In contrast, a positive bacterial culture and/or beneficial impact of antibiotic treatment may increase the probability of infection prostatitis to a high level and reduce the probability of inflammation and the probability of other conditions. Examples of this are shown in as shown in FIG. 30.

A total and calculation module can consolidate output from the separate probability generators for the three temporary prostate conditions: other temporary conditions, inflammation prostatitis, and infection prostatitis. Values are totaled for four variables: PSA and PSA increment, and free PSA and free PSA increment. Ratios are calculated for free PSA % (=free PSA/PSA) and free PSA increment % (=free PSA increment/PSA increment).

In an aspect, an elevated or increasing PSA trend is an indication that a long-term condition may be affecting the prostate. Methods herein can estimate the probability of these conditions using Bayesian processes.

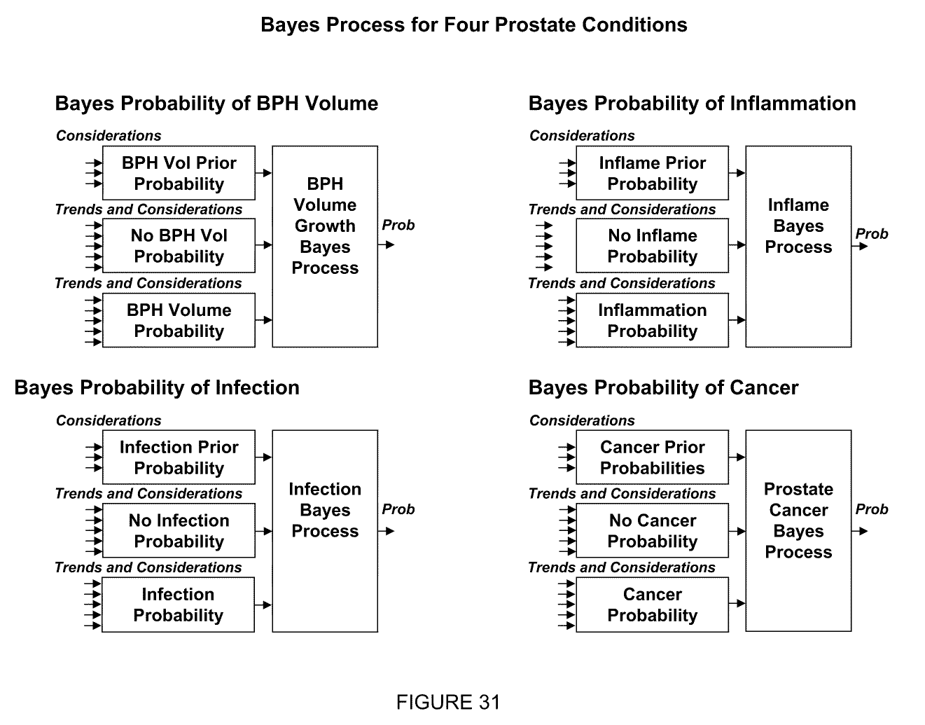

In an embodiment, four similar Bayes processes are used to calculate the probability of the prostate conditions: volume growth due to BPH, inflammation prostatitis, infection prostatitis and progressing cancer as shown in FIG. 31. The Bayes process uses three elements: the prior probability of the condition, the probability of the observed trend values conditional on all conditions and the probability of the observed trend values conditional on the absence of the condition but with all other conditions possible. Prior probabilities may be a function of age, race, genetics, demographics and other considerations.

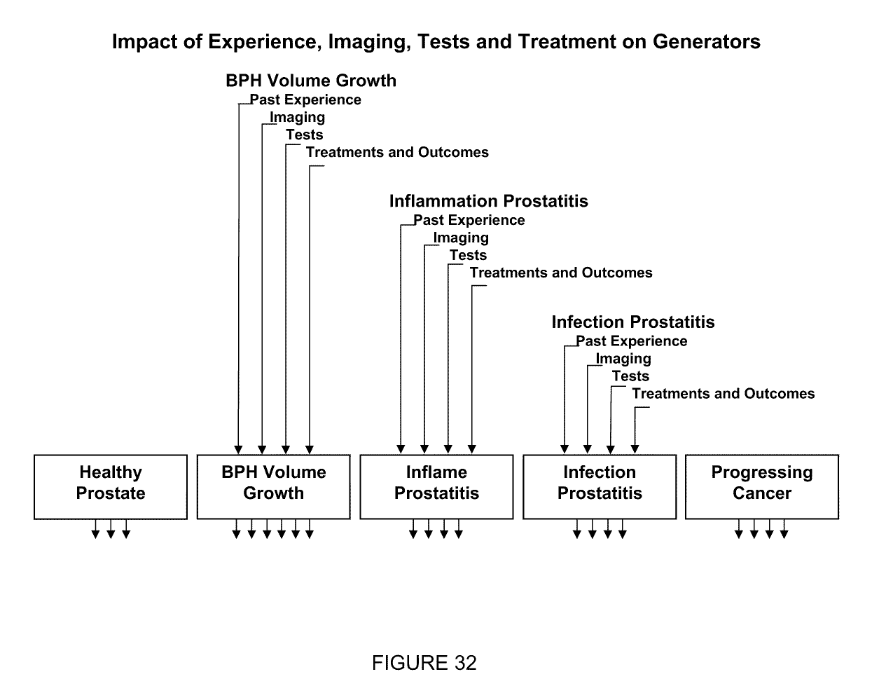

The probability distributions of each prostate condition can be affected by past experience and the results of imaging, tests, treatment and other medical procedures as shown in FIG. 32. For example, the prostate can be imaged using ultrasound or MRI equipment and its volume can be measured from the images. This measurement constrains the distributions of prostate volume, PSA and free PSA. For example, prostatic secretions can be cultured for bacterial infections. The results will affect the probability distributions produced by the infection prostatitis module. In a similar way, treatment with antibiotics can affect PSA levels. The outcome can affect the distributions produced by the infection prostatitis module. For example, a negative bacterial culture and no impact from antibiotic treatment will reduce the probability of infection prostatitis and increase the probability of other conditions, including progressing cancer. In contrast, a positive bacterial culture and/or beneficial impact of antibiotic treatment will increase the probability of infection prostatitis to a high level and reduce the probability of other conditions, including progressing cancer.

In an example, a total and calculation module consolidates output from the separate probability generators for the four benign prostate conditions: healthy prostate, volume growth due to BPH, inflammation prostatitis, infection prostatitis and progressing cancer. Values are totaled for six variables: prostate volume and volume velocity, PSA and PSA velocity, and free PSA and free PSA velocity. Ratios are calculated for free PSA % (=free PSA/PSA) and free PSA velocity % (=free PSA velocity/PSA velocity).

An enormous amount of data can be needed to define all the underlying distributions completely. In practice, the amount of data needed to define the distributions is not practical to obtain. Therefore, an iterative process is needed to tune the parameters of the underlying distributions so that known relationships are satisfied and the overall distributions conform to population studies.

In an aspect of the invention, an iterative Monte Carlo process generates multi-dimensional distributions for men of a given age without prostate cancer. Static parts of the distribution (no velocities as shown below) can be validated against available distributions. For example, the Center for Disease Control has published distributions of PSA, free PSA and free PSA % for U.S. men in ten year age ranges from age forty to age eighty and above, and the Mayo Clinic has published prostate volume and PSA distributions for men from age forty to age eighty in Olmsted County, Minn. Distributions like these constrain the overall distributions generated by the Monte Carlo process. Details of these distributions and other medical studies constrain the results of the specific probability generators and the relationships among them. For example, the CDC distributions show a significant decline in free PSA % for higher levels of PSA. This result strongly suggests that infection prostatitis accounts for an increasing proportion of higher PSA results because it is the only benign condition that produces free PSA in a percent that is significantly lower than the other benign conditions.

### Systems

In another aspect of the invention, a medical information system for assessing a disease of a subject is provided that comprises: an input device for receiving subject data; a processor that assesses a probability of said data relating to historical data; a storage unit in communication with the processor having a database for: (i) storing the subject data; (ii) storing historical data related to the disease; and an output device that transmits information relating to the probability of said data relating to historical data to an end user.

The invention also provides a method for assessing a disease in a subject comprising: collecting data from the subject corresponding to a biomarker for the disease at least two different times, wherein the data corresponding to the at least two different times form a trend; exporting said data for manipulation of said data by executing a method of the invention; and importing the results of said manipulation to an end user. For example, data is collected at a first location, such as a hospital, the data is exported to a second location, such as a remote server in any remote location, where a method of the invention is executed to obtain information regarding the disease in a subject, and then the information is imported from the remote location back to the first location, such as the point-of-care in the hospital, or the information is imported to a third location, such as a database.

It is further noted that the systems and methods may be implemented on various types of computer architectures, such as for example on a networked system or in a client-server configuration, or in an application service provider configuration, on a single general purpose computer or workstation. The systems and methods may include data signals conveyed via networks (for example, local area network, wide area network, internet, and combinations thereof), fiber optic medium, carrier waves, wireless networks for communication with one or more data processing devices. The data signals can carry any or all of the data disclosed herein (for example, user input data, the results of the analysis to a user) that is provided to or from a device.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform methods described herein.

The systems' and methods' data (for example, associations, mappings) may be stored and implemented in one or more different types of computer-implemented ways, such as different types of storage devices and programming constructs (for example, data stores, RAM, ROM, Flash memory, flat files, databases, programming data structures, programming variables, IF-THEN (or similar type) statement constructs). It is noted that data structures describe formats for use in organizing and storing data in databases, programs, memory, or other computer-readable media for use by a computer program.

The systems and methods may be provided on many different types of computer-readable media including computer storage mechanisms (for example, CD-ROM, diskette, RAM, flash memory, computer's hard drive, magnetic tape, and holographic storage) that contain instructions (for example, software) for use in execution by a processor to perform the methods' operations and implement the systems described herein.

The computer components, software modules, functions, data stores and data structures described herein may be connected directly or indirectly to each other in order to allow the flow of data needed for their operations. It is also noted that the meaning of the term module includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

In general, in yet another aspect, a computer readable medium is provided including computer readable instructions, wherein the computer readable instructions instruct a processor to execute step a) of the methods described above. The instructions can operate in a software runtime environment.

In general, in yet another aspect, a data signal is provided that can be transmitted using a network, wherein the data signal includes said posterior probability calculated in a step of the methods described above. The data signal can further include packetized data that is transmitted through wired or wireless networks.

In an aspect, a computer readable medium comprises computer readable instructions, wherein the instructions when executed carry out a calculation of the probability of a medical condition in a patient based upon data obtained from the patient corresponding to at least one biomarker. The computer readable instructions can operate in a software runtime environment of the processor. In an embodiment, a software runtime environment provides commonly used functions and facilities required by the software package. Examples of a software runtime environment include, but are not limited to, computer operating systems, virtual machines or distributed operating systems. As will be appreciated by those of ordinary skill in the art, several other examples of runtime environment exist. The computer readable instructions can be packaged and marketed as a software product or part of a software package. For example, the instructions can be packaged with an assay kit for PSA.

The computer readable medium may be a storage unit of the present invention as described herein. It is appreciated by those skilled in the art that computer readable medium can also be any available media that can be accessed by a server, a processor, or a computer. The computer readable medium can be incorporated as part of the computer-based system of the present invention, and can be employed for a computer-based assessment of a medical condition.

In an embodiment, the calculation of a probability can be carried out on a computer system. The computer system can comprise any or all of the following: a processor, a storage unit, software, firmware, a network communication device, a display, a data input, and a data output. A computer system can be a server. A server can be a central server that communicates over a network to a plurality of input devices and/or a plurality of output devices. A server can comprise at least one storage unit, such as a hard drive or any other device for storing information to be accessed by a processor or external device, wherein the storage unit can comprise one or more databases. In an embodiment, a database can store hundreds to millions of data points corresponding to a biomarker from hundreds to millions of subjects. A storage unit can also store historical data read from an external database or as input by a user. In an embodiment, a storage unit stores data received from an input device that is communicating or has communicated with the server. A storage unit can comprise a plurality of databases. In an embodiment, each of a plurality of databases corresponds to each of a plurality of prostate biomarkers. In another embodiment, each of a plurality of databases corresponds to each of a plurality of possible prostate conditions of a subject. An individual database can also comprise information for a plurality of possible medical conditions or a plurality of biomarkers or both. Further, a computer system can comprise multiple servers.

A processor can access data from a storage unit or from an input device to perform a calculation of an output from the data. A processor can execute software or computer readable instructions as provided by a user, or provided by the computer system or server. The processor may have a means for receiving patient data directly from an input device, a means of storing the subject data in a storage unit, and a means for processing data. The processor may also include a means for receiving instructions from a user or a user interface. The processor may have memory, such as random access memory, as is well known in the art. In one embodiment, an output that is in communication with the processor is provided.

After performing a calculation, a processor can provide the output, such as from a calculation, back to, for example, the input device or storage unit, to another storage unit of the same or different computer system, or to an output device. Output from the processor can be displayed by data display. A data display can be a display screen (for example, a monitor or a screen on a digital device), a print-out, a data signal (for example, a packet), an alarm (for example, a flashing light or a sound), a graphical user interface (for example, a webpage), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In an embodiment, an output device is the same device as the input device. Example output devices include, but are not limited to, a telephone, a wireless telephone, a mobile phone, a PDA, a flash memory drive, a light source, a sound generator, a fax machine, a computer, a computer monitor, a printer, an iPod, and a webpage. The user station may be in communication with a printer or a display monitor to output the information processed by the server.

A client-server, relational database architecture can be used in embodiments of the invention. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the invention, the server computer handles all of the database functionality. The client computer can have software that handles all the front-end data management and can also receive data input from users.

In an example, a subject or medical professional enters medical data from a prostate biomarker assay into a webpage. The webpage transmits the data to a computer system or server, wherein the data is stored and processed. For example, the data can be stored in databases the computer systems. Processors in the computer systems can perform calculations comparing the input data to historical data from databases available to the computer systems. The computer systems can then store the output from the calculations in a database and/or communicate the output over a network to an output device, such as a webpage or email. After a user has received an output from the computer system, the user can take a course of medical action according to the output. For example, if the user is a physician and the output is a probability of BPH above a threshold value, the physician can prescribe a pharmaceutical.

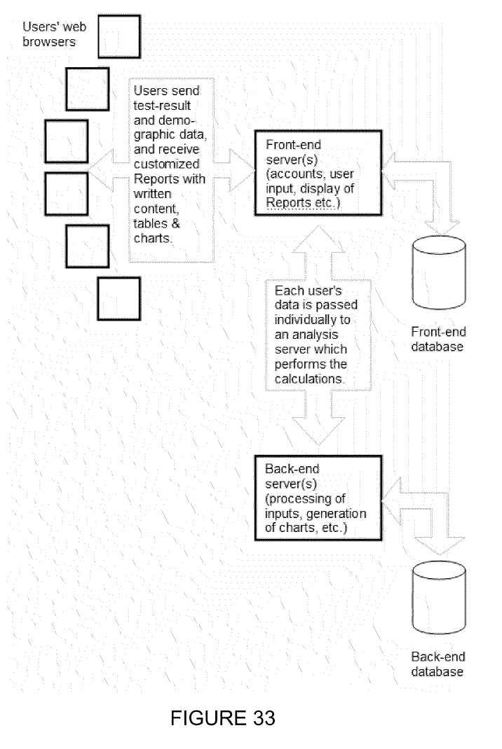

FIG. 33 demonstrates an example computer system of the invention. A set of users can use a web browser to enter data from a prostate biomarker assay into a graphical user interface of a webpage. The webpage is a graphical user interface associated with a front end server, wherein the front end server can communicate with the user's input device (for example, a computer) and a back end server. The front end server can either comprise or be in communication with a storage device that has a front-end database capable of storing any type of data, for example user account information, user input, and reports to be output to a user. Data from each user (for example, biomarker values and subject profiles) can be then be sent to a back end server capable of manipulating the data to generate a result. For example, the back end server can calculate a probability that a subject has a medical condition using the data input by the user. A back end server can comprise historical data relating to a prostatic condition to be evaluated, or a plurality of prostatic conditions. The back end server can then send the result of the manipulation or calculation back to the front end server where it can be stored in a database or can be used to generate a report. The results can be transmitted from the front end server to an output device (for example, a computer with a web browser) to be delivered to a user. A different user can input the data and receive the data. In an embodiment, results are delivered in a report. In another embodiment, results are delivered directly to an output device that can alert a user.

In an embodiment, a method of the invention comprises obtaining a sample from a subject, wherein the sample contains a biomarker. The sample can be obtained by the subject or by a medical professional. Examples of medical professionals include, but are not limited to, physicians, emergency medical technicians, nurses, first responders, psychologists, medical physics personnel, nurse practitioners, surgeons, dentists, and any other obvious medical professional as would be known to one skilled in the art. The sample can be obtained from any bodily fluid, for example, amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour. In an example, the sample is obtained by a blood draw, where the medical professional draws blood from a subject, such as by a syringe. The bodily fluid can then be tested to determine the prevalence of the biomarker. Biological markers, also referred to herein as biomarkers, according to the present invention include without limitation drugs, prodrugs, pharmaceutical agents, drug metabolites, biomarkers such as expressed proteins and cell markers, antibodies, serum proteins, cholesterol, polysaccharides, nucleic acids, biological analytes, biomarker, gene, protein, or hormone, or any combination thereof. At a molecular level, the biomarkers can be polypeptide, glycoprotein, polysaccharide, lipid, nucleic acid, and a combination thereof.

Information can be sent to a computer system automatically by a device that reads or provides the data from a biomarker assay. In another embodiment, information is entered by a user (for example, the subject or medical professional) into a computer system using an input device. The input device can be a personal computer, a mobile phone or other wireless device, or can be the graphical user interface of a webpage. For example, a webpage programmed in JAVA can comprise different input boxes to which text can be added by a user, wherein the string input by the user is then sent to a computer system for processing. The subject may input data in a variety of ways, or using a variety of devices. Data may be automatically obtained and input into a computer from another computer or data entry system. Another method of inputting data to a database is using an input device such as a keyboard, touch screen, trackball, or a mouse for directly entering data into a database.

In another embodiment, a system of the invention can further include a medical test for testing said subject for said prostate condition. The medical test can be a PSA assay. In yet another embodiment, a system can further include a medical treatment for treating said subject for said prostate condition.

In an embodiment, a computer system of the invention comprises a storage unit, a processor, and a network communication unit. For example, the computer system can be a personal computer, laptop computer, or a plurality of computers. The computer system can also be a server or a plurality of servers. Computer readable instructions, such as software or firmware, can be stored on a storage unit of the computer system. A storage unit can also comprise at least one database for storing and organizing information received and generated by the computer system. In an embodiment, a database comprises historical data, wherein the historical data can be automatically populated from another database or entered by a user.

In an embodiment, a processor of the computer system accesses at least one of the databases or receives information directly from an input device as a source of information to be processed. The processor can perform a calculation on the information source, for example, performing dynamic screening or a probability calculation method of the invention. After the calculation the processor can transmit the results to a database or directly to an output device. A database for receiving results can be the same as the input database or the historical database. An output device can communicate over a network with a computer system of the invention. The output device can be any device capable delivering processed results to a user. Example output devices include, but are not limited to, a telephone, a wireless telephone, a mobile phone, a PDA, a flash memory drive, a light source, a sound generator, a fax machine, a computer, a computer monitor, a printer, an iPOD, and a webpage.

An output of a computer system may assume any form, such as a computer program, webpage, or print-out. Any other suitable representation, picture, depiction or exemplification may be used.

Communication between devices or computer systems of the invention can be any method of digital communication including, for example, over the internet. Network communication can be wireless, ethernet-based, fiber optic, or through fire-wire, USB, or any other connection capable of communication as would be obvious to one skilled in the art. In an embodiment, information transmitted by a system or method of the invention can be encrypted by any method as would be obvious to one skilled in the art. In the field of medicine, or diagnostics, encryption may be necessary to maintain privacy of the data, as well as deter theft of information.

It is further noted that the systems and methods may include data signals conveyed via networks (for example, local area network, wide area network, internet), fiber optic medium, carrier waves, wireless networks for communication with one or more data processing or storage devices. The data signals can carry any or all of the data disclosed herein that is provided to or from a device.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform methods described herein. Other implementations may also be used, however, such as firmware or even appropriately designed hardware configured to carry out the methods and systems described herein.

The methods of the invention may be packaged as a computer program product, such as the expression of an organized set of instructions in the form of natural or programming language statements that is contained on a physical media of any nature (for example, written, electronic, magnetic, optical or otherwise) and that may be used with a computer or other automated data processing system of any nature (but preferably based on digital technology). Such programming language statements, when executed by a computer or data processing system, cause the computer system to act in accordance with the particular content of the statements. Computer program products include without limitation: programs in source and object code and/or test or data libraries embedded in a computer readable medium. Furthermore, the computer program product that enables a computer system or data processing equipment device to act in preselected ways may be provided in a number of forms, including, but not limited to, original source code, assembly code, object code, machine language, encrypted or compressed versions of the foregoing and any and all equivalents.

Information before, after, or during processing can be displayed on any graphical display interface in communication with a computer system (for example, a server). A computer system may be physically separate from the instrument used to obtain values from the subject. In an embodiment, a graphical user interface also may be remote from the computer system, for example, part of a wireless device in communication with the network. In another embodiment, the computer and the instrument are the same device.

An output device or input device of a computer system of the invention can include one or more user devices comprising a graphical user interface comprising interface elements such as buttons, pull down menus, scroll bars, fields for entering text, and the like as are routinely found in graphical user interfaces known in the art. Requests entered on a user interface are transmitted to an application program in the system (such as a Web application). In one embodiment, a user of user device in the system is able to directly access data using an HTML interface provided by Web browsers and Web server of the system.

A graphical user interface may be generated by a graphical user interface code as part of the operating system or server and can be used to input data and/or to display input data. The result of processed data can be displayed in the interface or a different interface, printed on a printer in communication with the system, saved in a memory device, and/or transmitted over a network. A user interface can refer to graphical, textual, or auditory information presented to a user and may also refer to the control sequences used for controlling a program or device, such as keystrokes, movements, or selections. In another example, a user interface may be a touch screen, monitor, keyboard, mouse, or any other item that allows a user to interact with a system of the invention as would be obvious to one skilled in the art.

### Medical Actions

In general, in yet another aspect, a method of taking a course of medical action by a user is provided including initiating a course of medical action based on a probability delivered from an output device to said user.

The course of medical action can be delivering medical treatment to said subject. The course of medical action can include, for example, administration of medical tests, medical imaging of said subject, setting a specific time for delivering medical treatment, a biopsy, and a consultation with a medical professional.

A method can further include diagnosing the prostatic condition of the subject by said user with a probability from the methods or systems disclosed herein.

A system or method of the invention can involve delivering a medical treatment or initiating a course of medical action. If a disease has been assessed or diagnosed by a method or system of the invention, a medical professional can evaluate the assessment or diagnosis and deliver a medical treatment according to his evaluation. Medical treatments can be any method or product meant to treat a disease or symptoms of the disease. In an embodiment, a system or method initiates a course of medical action. A course of medical action is often determined by a medical professional evaluating the results from a processor of a computer system of the invention. For example, a medical professional may receive output information that informs him that a subject has a 97% probability of having a particular medical condition. Based on this probability, the medical professional can choose the most appropriate course of medical action, such as biopsy, surgery, medical treatment, or no action. In an embodiment, a computer system of the invention can store a plurality of examples of courses of medical action in a database, wherein processed results can trigger the delivery of one or a plurality of the example courses of action to be output to a user. In an embodiment, a computer system outputs information and an example course of medical action. In another embodiment, the computer system can initiate an appropriate course of medical action. For example, based on the processed results, the computer system can communicate to a device that can deliver a pharmaceutical to a subject. In another example, the computer system can contact emergency personnel or a medical professional based on the results of the processing.

The medical action can be based on rules imposed by the medical professional or the computer system. Courses of medical action include, but are not limited to, surgery, radiation therapy, chemotherapy, prescribing a medication, evaluating mental state, delivering pharmaceuticals, monitoring or observation, biopsy, imaging, and performing assays and other diagnostic tests. In an embodiment, the course of medical action may be inaction. Medical action also includes, but is not limited to, ordering more tests performed on the patient, administering a therapeutic agent, altering the dosage of an administered therapeutic agent, terminating the administration of a therapeutic agent, combining therapies, administering an alternative therapy, placing the subject on a dialysis or heart and lung machine, performing computerized axial tomography (CAT or CT) scan, performing magnetic resonance imaging (MRI), performing a colonoscopy, administering a pain killer, prescribing a medication. In some embodiments, the subject may take medical action.

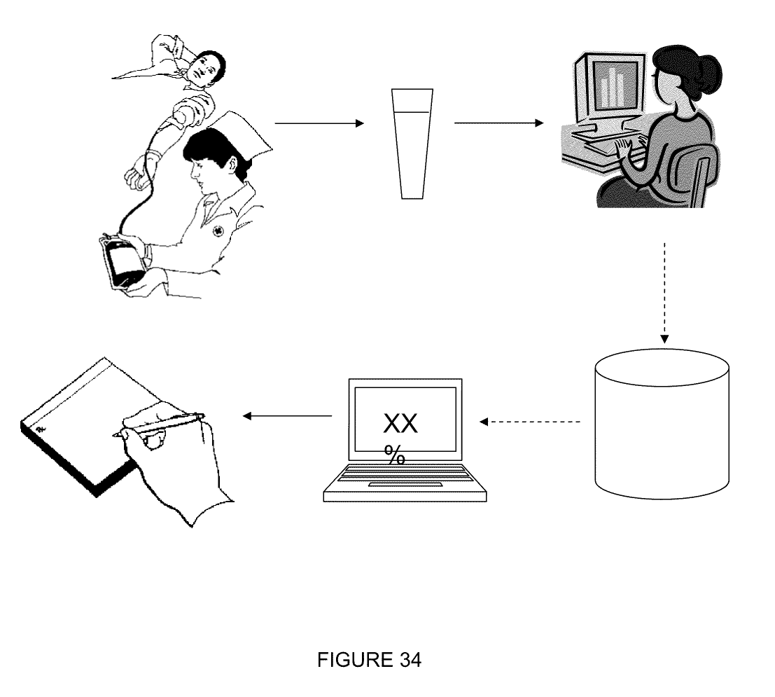

FIG. 34 illustrates a method of delivering a probability that a subject has a medical condition to a user and using the probability to take a course of medical action. A blood sample is drawn from a patient by a medical professional. In other embodiments, any method of obtaining a biomarker values from a subject may be used as would be obvious to one skilled in the art, such as swabs and urine tests. In FIG. 34, the sample is assayed for a biomarker and biomarker values are generated. As described herein, there may be many suitable methods for generating and obtaining biomarker values. The values can then input into a computer by a medical professional or other user, such as the subject or an assistant. The data can then be processed by a method of the invention to calculate the probability that a subject has the medical condition. An output is generated and delivered to a user on a computer monitor, for example, the output delivers the probability of a subject having a medical condition and is display on a personal computer or laptop of the subject's doctor. The output can also be delivered to the subject himself or to a different medical professional. In another embodiment, the output is delivered to a notification system, such as an alarm or another computer-based program. In FIG. 34, based on the output, a physician can take a medical action as described herein. In this example, the output initiates a medical professional writing a prescription.

As new technology is developed to deliver blood test results at the point of care within a short time, perhaps minutes, in an embodiment of the invention the device doing the test can communicate to a computer system wirelessly, through a docking station or other physical link or by other means, including manual entry of the results. The computer system can have software and/or a storage medium that receives the test results and other information about the patient and for performing a calculation method of the invention. A computer system can be on remote servers that can process the new data along with other patient information already stored in the system. Parallel processing can be used to analyze the data and create a report quickly, perhaps in minutes. A report can be transmitted to the computer in the doctor's office for viewing on screen or for printing and use as hard copy. For example, the doctor may review the report with the patient and decide on a course of medical action. For example, the doctor and patient may decide on ultrasound imaging to measure the volume of the patient's prostate. The prostate volume measured can be entered into a computer system for further analysis that can then create a new report that can be transmitted to the doctor's display for viewing or printing. For example, the doctor may review the new results with the patient and decide on a new course of medical action. For example, the doctor and patient may decide to culture prostate secretions for infection and start a course of antibiotics to treat the possible infection.

In another embodiment with new technology developed to deliver automated blood tests for a variety of biomarkers at one time, automated protein profile equipment reports the levels of a wide variety of proteins and other biomarkers in a sample. Biomarker values can be automatically uploaded to a computer system as described herein and can be added to other patient information already stored in the system. For example, new probabilities can be calculated for all medical conditions being considered. The doctor and/or the patient can consider the results and choose appropriate courses of medical action.

Individuals can vary in their predisposition for various conditions. In an embodiment, methods of the invention incorporate these predispositions or risk factors into the prior probabilities of each condition for each individual. For example, genetic testing might show a man has a three times higher than normal risk of prostate disease. Family history or race might suggest other men have a two times higher than normal risk of prostate cancer. Several risk factors can be combined into an overall risk ratio that reflects a person increased or reduced risk of a condition compared to an overall population. Risk factors can include without limitation: gene profile, family history, race, obesity (BMI), physical condition, geographic location of home and work over time, diet and exercise regimen, exposure to environmental factors and other things.

An individual's future predisposition to various conditions can depend on their past incidence of that condition and other related conditions. In another embodiment, methods of the invention incorporate these predispositions or risk factors into either the prior probabilities of each condition for each individual or an explicit algorithm that may be a Bayes process. For example, a man with a history of prostatitis caused by infection has an increased risk of that condition in the future. If a prior probability is adjusted then algorithms are used to combine the risk factor based on past history with other risk factors into an overall risk ratio that reflects a person increased or reduced risk of a condition compared to an overall population. Alternatively, a different algorithm can be used to calculate a new posterior probability of a condition based on the details of the past history of that condition and related conditions, perhaps using a Bayes process.

It is to be understood that the exemplary methods and systems described herein may be implemented in various forms of hardware, software, firmware, special purpose processors, or a combination thereof. Preferably, a calculation method of the present invention is implemented in software as an application program tangibly embodied on one or more program storage devices. The application program may be executed by any machine, device, or platform comprising suitable architecture. It is to be further understood that, because some of the systems and methods depicted in the Figures are preferably implemented in software, the actual connections between the system components (or the process steps) may differ depending upon the manner in which the method is programmed. Given the teachings herein, one of ordinary skill in the related art will be able to contemplate or practice these and similar implementations or configurations of the present invention.

### Business Methods

A business method as described herein can comprise suggesting a course of medical action to said user based on said posterior probabilities, and the suggestion can be provided for a fee.

In an embodiment of a business method of the invention, a posterior probability of a medical condition is delivered to a user, wherein the user, without limitation, is a patient, a medical person (such as a physician), a health systems, or a lab. For example, a subject can have a blood test that is assayed in a lab or at the point-of-care and then a user sends the information from the assay to company (such as over the internet), where the company performs processes or calculations with the information and delivers an output (such as a probability of the occurrence of a disease) to the user. The company can provide the output for a fee.

In an embodiment, a business method comprises selling services of the calculations and delivery of information directly to patients. For example, the patients can use this information with their physicians.

With respect to this disclosure, while examples have been used to disclose the invention, including the best mode, and also to enable any person skilled in the art to make and use the invention, the patentable scope of the invention is defined by claims, and may include other examples that occur to those skilled in the art. Accordingly the examples disclosed herein are to be considered non-limiting. As an illustration, it should be understood that for the processing flows described herein, the steps and the order of the steps may be altered, modified, removed, and/or augmented and still achieve the desired outcome.

What is claimed is:

1. A method of detecting prostate enlargement comprising:
a) monitoring a prostate biomarker value at one or more times;
b) measuring a volume of the prostate of the subject when the prostate biomarker value is or exceeds a target value;
c) calibrating the prostate biomarker value with the volume measurement; and
d) detecting the presence or absence of prostate enlargement in the subject by measuring the prostate biomarker value.

2. The method of claim 1, wherein the calibrating step comprises determining the prostate biomarker value density.

3. The method of claim 1, wherein the prostate biomarker is PSA or fPSA.

4. The method of claim 1 further comprising monitoring the prostate biomarker value two or more times, thereby forming a prostate biomarker trend.

5. The method of claim 4, wherein the monitoring step comprises eliminating anomalous prostate biomarker values from the trend that are values outside of a tolerance.

6. The method of claim 1, wherein the measuring step comprises an ultrasound measurement.

7. The method of claim 1 further comprising calculating the probability of prostate enlargement from the prostate biomarker value.

8. A method of performing a course of medical action for prostate enlargement in a subject, the method comprising:
a) monitoring a prostate biomarker value from a subject at one or more times;
b) measuring a volume of the prostate of the subject when the prostate biomarker value is or exceeds a target value;
c) calibrating the prostate biomarker value with the volume measurement;
d) estimating the volume of the prostate by measuring the prostate biomarker value at a time after the measuring step;
e) performing a course of medical action when the estimated volume of the prostate is greater than a threshold value.

9. The method of claim 8, further comprising repeating steps d) and e) after determining the outcome of the course of medical action.

10. The method of claim 8, wherein steps c) and d) are performed by a computer system.

11. The method of claim 10, wherein said computer system comprises a device for network communication, a storage unit, and a processor.

12. The method of claim 10, wherein said computer system comprises a Monte Carlo engine.

13. The method of claim 8, further comprising calculating the probability of prostate enlargement from the prostate biomarker value.

14. The method of claim 8, wherein the calibrating step comprises determining the prostate biomarker value density.

15. The method of claim 8, wherein the prostate biomarker is PSA or fPSA.

16. The method of claim 8, further comprising monitoring the prostate biomarker value two or more times, thereby forming a prostate biomarker trend.

17. The method of claim 17, wherein the monitoring step comprises eliminating anomalous prostate biomarker values from the trend that are values outside of a tolerance.

18. The method of claim 8, wherein the measuring step comprises an ultrasound measurement.

19. The method of claim 8, wherein the course of medical action is delivering medical treatment to said subject.

20. The method of claim 19, wherein the medical treatment is selected from a group consisting of the following: a pharmaceutical, TURP, TUNA, and TUMT.

21. The method of claim 8, wherein the course of medical action comprises administration of medical tests.

22. The method of claim 8, wherein the course of medical action comprises medical imaging of said subject.

23. The method of claim 8, wherein the course of medical action comprises a consultation with a medical professional.

24. The method of claim 8, further comprising performing a digital rectal exam and calibrating the prostate biomarker value with the digital rectal exam results and the volume measurement.

25. A method comprising:
a) establishing a Bayesian network comprising a plurality of prostatitis conditions of the prostate, wherein the priors of the Bayesian network are provided by historical data;
b) calculating a probability of a target prostatitis condition of a prostate using a trend of a prostate biomarker value over time;
c) providing the probability of the target medical condition of the prostate to the Bayesian network as a prior for the target medical condition;
d) performing a course of medical action in respect to the target medical condition and receiving the result of the course of medical action;
e) adjusting the priors of the Bayesian network according to the result; and
f) calculating the probability of the target prostatitis condition.

26. The method of claim 25, wherein the target prostatitis condition is inflammation or infection.

27. The method of claim 25, wherein the course of medical action is delivering an anti-inflammatory.

28. The method of claim 25, wherein the course of medical action is delivering an anti-biotic.

29. The method of claim 25, wherein the course of medical action is performing a white blood cell count.

30. The method of claim 25, wherein the course of medical action is culturing prostate secretions.

31. The method of claim 25, further comprising detecting a change in the prostate biomarker value after step d).

32. The method of claim 31, wherein the change demonstrates an effectiveness of a course of medical action.

33. The method of claim 25 further comprising calculating the probability of prostate cancer using the probability of the target prostatitis condition calculated in step f).

34. A method of providing a medical treatment of a prostate condition comprising:
a) providing a course of medical action to a subject with a prostate condition;
b) detecting a change in a prostate biomarker value of the subject before providing the medical treatment and during the medical treatment; and
c) adjusting the course of medical action based upon the change in the prostate biomarker value.

35. The method of claim 34, wherein the prostate biomarker is PSA or fPSA.

36. The method of claim 34, further comprising detecting a change in the prostate biomarker value at more than one time during treatment.

37. The method of claim 34, wherein the prostate biomarker over time forms a prostate biomarker trend and wherein the change in the prostate biomarker value is detected by a change in the prostate biomarker trend.

38. The method of claim 34, wherein the prostate condition is BPH.

39. The method of claim 38, wherein the course of medical action is delivering a pharmaceutical to the subject, and the adjusting of the course of medical action comprises adjusting at least one of the following: treatment time or treatment dose.

40. The method of claim 38, wherein the course of medical action is delivering a pharmaceutical to the subject, and the adjusting of the course of medical action comprises changing the pharmaceutical being delivered during treatment.

41. The method of claim 34, wherein the prostate condition is prostatitis infection.

42. The method of claim 41, wherein the course of medical action is delivering an antibiotic to the subject, and the adjusting of the course of medical action comprises adjusting at least one of the following: treatment time or treatment dose.

43. The method of claim 41, wherein the course of medical action is delivering a pharmaceutical to the subject, and the adjusting of the course of medical action comprises changing the pharmaceutical being delivered during treatment.

44. The method of claim 34, wherein the prostate condition is prostatitis inflammation.

45. The method of claim 44, wherein the course of medical action is delivering an anti-inflammatory to the subject, and the adjusting of the course of medical action comprises adjusting at least one of the following: treatment time or treatment dose.

46. The method of claim 44, wherein the course of medical action is delivering a pharmaceutical to the subject, and the adjusting of the course of medical action comprises changing the pharmaceutical being delivered during treatment.
